(19) United States
(12) Patent Application Publication
(10) Pub. No.: US 2024/0384330 A1
(43) Pub. Date: Nov. 21, 2024

(54) COVALENTLY LINKED BRANCHED DNA STRUCTURES AND USES THEREOF

(71) Applicant: 10x Genomics, Inc., Pleasanton, CA (US)

(72) Inventors: Marco MIGNARDI, Sundbyberg (SE); Toon VERHEYEN, Solna (SE)

(21) Appl. No.: 18/658,702

(22) Filed: May 8, 2024

Related U.S. Application Data
(60) Provisional application No. 63/465,245, filed on May 9, 2023.

(51) Int. Cl. C12Q 1/6825; C12Q 1/6806; C12Q 1/6876

(52) U.S. Cl. CPC C12Q 1/6825; C12Q 1/6806; C12Q 1/6876

(57) ABSTRACT

The present disclosure relates in some aspects to methods and compositions for analyzing a biological sample with a covalently linked branched nucleic acid (bNA) structure. In some aspects, covalently linked bNA structures are useful for methods of in situ analysis, including for detecting nucleic acid analytes or non-nucleic acid analytes associated with labeling agents comprising nucleic acids.

### CROSS-REFERENCE TO RELATED APPLICATIONS

This application claims priority to U.S. provisional Patent Application No. 63/465,245, filed May 9, 2023, entitled “COVALENTLY LINKED BRANCHED DNA STRUCTURES AND USES THEREOF”, which is herein incorporated by reference in its entirety for all purposes.

### FIELD

The present disclosure relates in some aspects to branched nucleic acid structures for transcriptomic profiling in situ.

### BACKGROUND

Genomic, transcriptomic, and proteomic profiling of cells and tissue samples using microscopic imaging can resolve multiple analytes of interest at the same time, thereby providing valuable information regarding analyte abundance and localization in situ. Thus, in situ assays are important tools, for example, for understanding the molecular basis of cell identity and developing treatment for diseases. In some aspects, existing methods are limited by the ability to provide sufficient signal amplification for efficient detection. Provided herein are methods and compositions that address such and other needs.

### SUMMARY

In some aspects, provided herein are covalently linked branched DNA (bDNA) structures and methods of assembling covalently linked bDNA structures via interstrand crosslinking or formation of covalent linkage points (e.g., using click chemistry). In some aspects, provided herein are methods of analyzing a biological sample using the bDNA structures. In some embodiments, the bDNA structures are used to detect a target nucleic acid in the biological sample (e.g., a nucleic acid analyte such as an RNA, or a target nucleic acid associated with a nucleic acid or non-nucleic acid analyte). In some embodiments, detectably labeled probes or detectable moieties are covalently linked to the bDNA structure. In some aspects, the covalent linkages in the bDNA structure improve the stability of the bDNA structure and/or the signal intensity of the bDNA structure.

In some aspects, provided herein is a method for analyzing a biological sample, comprising: (a) contacting the biological sample with a crosslinked branched nucleic acid (bNA) structure comprising a first nucleic acid strand and a plurality of second nucleic acid strands, wherein the first nucleic acid strand comprises: (i) an adapter hybridization region that hybridizes to an adapter region in a target nucleic acid or in a probe or probe set hybridized to a target nucleic acid in the biological sample, and (ii) a plurality of branch hybridization regions (BHR), wherein each of the second nucleic acid strands comprises a complementary branch hybridization region (BHR′) and a detectable label or an overhang region comprising one or more reporter regions (e.g., to be detected by sequencing or for directly or indirectly binding to one or more detectably labeled probes), and wherein BHR′ hybridizes to BHR, wherein at least a subset of the second nucleic acid strands are covalently attached to the first nucleic acid strand via an interstrand crosslink in the branch hybridization region; and (b) detecting the hybridized bNA structure at a location in the biological sample, thereby detecting the target nucleic acid at the location in the biological sample.

In some embodiments, the adapter region is in a probe or probe set, and the method comprises contacting the biological sample with the probe or probe set, wherein the probe or probe set comprises (i) a recognition sequence that hybridizes to the target nucleic acid in the biological sample, and (ii) an overhang region comprising the adapter region.

In some embodiments, each of the second nucleic acid strands comprises an overhang region comprising a plurality of reporter regions, wherein the bNA structure comprises a plurality of detectably labeled probes hybridized to the reporter regions via complementary reporter hybridization regions. In some embodiments, at least a subset of the reporter regions are covalently attached to the hybridized detectably labeled probes via an interstrand crosslink.

In some embodiments, each of the second nucleic acid strands may be covalently linked to a plurality of detectable moieties at a plurality of linkage points. In some embodiments, the linkage points comprises triazole linkages. In some embodiments, the linkage points comprises thiol linkages. In some embodiments, the linkage points are formed by amine-reactive chemistry. In some embodiments, wherein each of the linkage points are separated by at least 10 nucleotide residues. In some embodiments, each of the linkage points are separated by between about 10 and about 20 nucleotide residues.

In some embodiments, the second nucleic acid strand comprises an overhang region functionalized with a plurality of acceptor moieties, wherein the method comprises covalently attaching the detectable moieties to the acceptor moieties to form the plurality of linkage points.

In some embodiments, the acceptor moieties can comprise dibenzocyclooctyne (DBCO)- or alkyne-modified bases and the detectable moieties can comprise an azide moiety; or the acceptor moieties can comprise azide-modified bases and the detectable moieties can comprise a DBCO or alkyne moiety. In some embodiments, covalently attaching the detectable moieties may comprise performing a copper-free or copper-catalyzed click chemistry reaction.

In some embodiments, prior to the contacting of the biological sample with the crosslinked bNA structure, the method comprises generating the crosslinked bNA structure by providing a mixture comprising the first nucleic acid strand and the plurality of second nucleic acid strands and allowing the branch hybridization regions (BHR) to hybridize to the complementary branch hybridization region (BHR′), and irradiating the mixture to generate interstrand crosslinks between the branch hybridization regions (BHR) and the complementary branch hybridization region (BHR′). In some embodiments, generating the crosslinked bNA structure further comprises contacting the second nucleic acid strand with the plurality of detectably labeled probes and allowing the detectably labeled probes to hybridize to the reporter regions, and irradiating the mixture to generate interstrand crosslinks between the reporter regions and the hybridized detectably labeled probes.

In some embodiments, the method comprises detecting the reporter region in a bNA structure by sequencing all or a portion of the reporter region. In some embodiments, the method comprises detecting a reporter region in a bNA structure by sequencing all or a portion of the reporter region at a location in the biological sample. In some embodiments, the method comprises contacting the bNA structure with a sequencing primer that binds adjacent to the reporter region. In some embodiments, detecting the hybridized bNA structure is performed by sequencing e.g., sequencing-by-synthesis (SBS), sequencing-by-avidity (SBA), or sequencing-by-binding (SBB).

In some embodiments, the interstrand crosslink between the branch hybridization region (BHR) and the complementary branch hybridization region (BHR′) is generated by irradiating a crosslinkable moiety comprised by the branch hybridization region (BHR) and/or the complementary branch hybridization region (BHR′). In some embodiments, the interstrand crosslinks may occur at internal nucleotides of the BHR and BHR′.

In some embodiments, the interstrand crosslink between the reporter region and the complementary reporter hybridization region is generated by irradiating a crosslinkable moiety comprised by the reporter region and/or the complementary reporter hybridization region. In some embodiments, the interstrand linkage is generated by UV-irradiation of a photocrosslinkable intercalating agent. In some embodiments, the interstrand linkage is generated by UV-irradiation of a crosslinkable moiety. In some embodiments, the crosslinkable moiety is a modified nucleoside in the first nucleic acid strand, second nucleic acid strands, and/or detectably labeled probes.

In some embodiments, the method comprises irradiating the mixture to photo-activate the crosslinkable moiety. In some embodiments, the method comprises irradiating the mixture to photo-activate the intercalating agent. In some embodiments, the mixture is irradiated using a 350-400 nm wavelength of light. In some embodiments, the mixture is irradiated using a 100-280 nm wavelength of light.

In some embodiments, the crosslinkable moiety is 5-bromo-2′-deoxyuridine (BrdU) or 5-bromo-2′-deoxycytidine (BrdC). In some embodiments, the crosslinkable moiety is a vinylcarbazone-based moiety. In some embodiments, the crosslinkable moiety is a 3-cyanovinylcarbazole phosphoramidite or a pyranocarbazole phosphoramidite. In some embodiments, the crosslinkable moiety is a 3-cyanovinylcarbazole (^{CNV}K) nucleoside, a 3-cyanovinylcarbazole modified D-threoninol (^{CNV}D), a pyranocarbazole nucleoside (^{PC}X) or a pyranocarbazole modified D-threoninol (^{PCX}D). In some embodiments, wherein the crosslinkable moiety is a psoralen or a psoralen derivative. In some embodiments, the psoralen is a C2 psoralen. In some embodiments, the crosslinkable moiety is a psoralen C2 phosphoramidite. In some embodiments, the crosslinkable moiety is a coumarin. In some embodiments, the crosslinkable moiety is a 5′-Dimethoxytrityl-2′-deoxy-4-(2-cyanoethylthio)-Thymidine,3′-[(2-cyanoethyl)-(N,N-diisopropyl)]-phosphoramidite (4-Thio-dT-CE phosphoramidite). In some embodiments, the crosslinkable moiety is a 5′-Dimethoxytrityl-5-iodo-2′-deoxyUridine,3′-[(2-cyanoethyl)-(N,N-diisopropyl)]-phosphoramidite (5-I-dU-CE phosphoramidite).

In some embodiments, the interstrand crosslink is reversible. In some embodiments, after detecting the hybridized bNA structure, the method comprises irradiating the biological sample to reverse one or more of the interstrand linkages. In some embodiments, irradiating the biological sample to reverse one or more of the interstrand linkages comprises irradiating the sample with a wavelength between about 300 nm and about 320 nm. In some embodiments, irradiating the biological sample to reverse one or more of the interstrand linkages comprises irradiating the sample with a wavelength between about 305 nm and about 312 nm. In some embodiments, irradiating the biological sample to reverse one or more of the interstrand linkages comprises irradiating the sample with a wavelength between about 250 nm and about 300 nm. In some embodiments, irradiating the biological sample to reverse one or more of the interstrand linkages comprises irradiating the sample with a wavelength of about 254 nm.

In some embodiments, detecting the bNA structure comprises imaging the biological sample at one or more excitation wavelengths between about 488 nm and about 647 nm. In some embodiments bNA structure is stable during the imaging.

In some embodiments, the method comprises imaging the biological sample at an excitation wavelength between 312 nm and 358 nm to detect a nuclear stain. In some embodiments, the nuclear stain is DAPI. In some embodiments, reversing one or more of the interstrand linkages occurs during the imaging of the nuclear stain. In some embodiments, imaging the biological sample at an excitation wavelength between 312 nm and 358 nm is performed after detecting the bNA structure at the location in the biological sample.

In some embodiments, the method comprises washing the biological sample to remove the bNA structure after detecting the bNA structure at the location in the biological sample. In some embodiments, one or more of the interstrand linkages are reversed during or prior to the washing.

In some aspects, provided herein is a method for analyzing a biological sample, comprising: (a) contacting the biological sample with a branched nucleic acid (bNA) structure comprising a first nucleic acid strand covalently connected to a plurality of second nucleic acid strands at a plurality of branch points, wherein each of the second nucleic acid strands is covalently linked to a plurality of detectable moieties at a plurality of triazole linkage points, and wherein the first nucleic acid strand comprises an adapter hybridization region that hybridizes to an adapter region in a target nucleic acid or in a probe or probe set hybridized to a target nucleic acid in the biological sample; and (b) detecting the bNA structure at a location in the biological sample, thereby detecting the target nucleic acid at the location in the biological sample.

In some embodiments, each of the linkage points are separated by a linker of at least 5 nm in length. In some embodiments, each of the linkage points are separated by at least 10 nucleotide residues. In some embodiments, each of the linkage points are separated by between about 10 and about 20 nucleotide residues. In some embodiments, the plurality of triazole linkage points is in an overhang region of the second nucleic acid strand. In some embodiments, the method comprises covalently attaching the detectable moieties to a plurality of acceptor moieties in the second nucleic acid strand to form the plurality of linkage points.

In some embodiments, the acceptor moieties comprises dibenzocyclooctyne (DBCO)- or alkyne-modified bases and the detectable moieties can comprise an azide moiety; or the acceptor moieties comprises azide-modified bases and the detectable moieties comprises a DBCO or alkyne moiety. In some embodiments, covalently attaching the detectable moieties comprises performing a first copper-free or copper-catalyzed click chemistry reaction. In some embodiments, the plurality of branch points comprises triazole linkages. In some embodiments, the plurality of branch points comprises thiol linkages. In some embodiments, the plurality of branch points is formed by amine-reactive chemistry. In some embodiments, each of the plurality of branch points are separated by at least 10 nucleotide residues. In some embodiments, each of the plurality of branch points are separated by between about 10 and about 20 nucleotide residues.

In some embodiments, the first nucleic acid strand comprises an overhang region functionalized with a plurality of first attachment moieties and each of the second nucleic acid strands is functionalized with a second attachment moiety, wherein the method comprises covalently attaching the first attachment moieties to the second attachment moieties to form the plurality of branch points.

In some embodiments, the first attachment moieties comprises dibenzocyclooctyne (DBCO)- or alkyne-modified bases and the second attachment moiety can comprise an azide moiety; or the first attachment moieties can comprise azide-modified bases and the second attachment moiety can comprise a DBCO or alkyne moiety. In some embodiments, covalently attaching the first attachment moieties to the second attachment moieties comprises performing a second copper-free or copper-catalyzed click chemistry reaction. In some embodiments, the second attachment moiety may not react with the detectable moieties. In some embodiments, the first and second copper-free or copper-catalyzed click chemistry reactions are performed sequentially in either order.

In some embodiments, the bNA structure is a first bNA structure, and detecting the first bNA structure comprises detecting a first signal of a signal code assigned to the target nucleic acid, wherein after detecting the hybridized first bNA structure at a location in the biological sample, the method comprises: (c) removing and/or cleaving the first bNA structure, (d) contacting the biological sample with a second bNA structure, wherein the second bNA structure binds directly or indirectly to the target nucleic acid, and (e) detecting the second bNA structure, wherein detecting the second bNA structure comprises detecting a second signal of the signal code assigned to the target nucleic acid.

In some embodiments, removing the first bNA structure comprises de-crosslinking the first bNA structure. In some embodiments, cleaving the first bNA structure comprises cleaving off the detectable label or cleaving the bNA structure to remove the detectable label or detectably labeled probes.

In some embodiments, the target nucleic acid is a cellular nucleic acid analyte or a product thereof. In some embodiments, the target nucleic acid is associated with a non-nucleic acid analyte. In some embodiments, the target nucleic acid is an oligonucleotide reporter in a labeling agent that binds to the analyte. In some embodiments, the target nucleic acid is RNA. In some embodiments, the target nucleic acid is mRNA. In some embodiments, the target nucleic acid is an RNA fragment.

In some embodiments, the target nucleic acid is a rolling circle amplification product. In some embodiments, the method comprises generating the rolling circle amplification product from a circular or circularized probe or probe set associated with an analyte in the biological sample. In some embodiments, the analyte is a nucleic acid analyte and the circular or circularized probe or probe set may be associated with the analyte by binding directly or indirectly to the nucleic acid analyte. In some embodiments, the analyte is a non-nucleic acid analyte and the circular or circularized probe or probe set may be associated with the analyte by binding directly or indirectly to an oligonucleotide reporter in a labeling agent that binds to the analyte.

In some embodiments, the biological sample is non-homogenized. In some embodiments, the biological sample is selected from the group consisting of a formalin-fixed, paraffin-embedded (FFPE) sample, a frozen tissue sample, and a fresh tissue sample. In some embodiments, the biological sample may be permeabilized. In some embodiments, the biological sample may be embedded in a matrix. In some embodiments, the matrix comprises a hydrogel. In some embodiments, the biological sample may be cleared. In some embodiments, the biological sample is a tissue slice between about 1 μm and about 50 μm in thickness. In some embodiments, the tissue slice is between about 5 μm and about 35 μm in thickness.

In some aspects, provided herein is a composition comprising a branched DNA (bDNA) structure comprising a first nucleic acid strand and a plurality of second nucleic acid strands, wherein the first nucleic acid strand comprises: (i) an adapter hybridization region, and (ii) a plurality of branch hybridization regions (BHR), wherein each of the second nucleic acid strands comprises a complementary branch hybridization region (BHR′) and a detectable label or an overhang region comprising one or more reporter regions for directly or indirectly binding to one or more detectably labeled probes, and wherein BHR′ hybridizes to BHR, wherein at least a subset of the second nucleic acid strands are covalently attached to the first nucleic acid strand via an interstrand crosslink in the branch hybridization region.

In some aspects, provided herein is a composition, comprising a branched nucleic acid (bNA) structure comprising a first nucleic acid strand covalently connected to a plurality of second nucleic acid strands at a plurality of branch points, wherein each of the second nucleic acid strands is covalently linked to a plurality of detectable moieties at a plurality of triazole linkage points.

In some aspects, provided herein is a kit, comprising: a) at least two different species of branched DNA structures, wherein each species of branched DNA structure comprises: a first nucleic acid strand and a plurality of second nucleic acid strands, wherein the first nucleic acid strand comprises: (i) an adapter hybridization region, and (ii) a plurality of branch hybridization regions (BHR), wherein each of the second nucleic acid strands comprises a complementary branch hybridization region (BHR′) and a detectable label or an overhang region comprising one or more reporter regions for directly or indirectly binding to one or more detectably labeled probes, and wherein BHR′ hybridizes to BHR, wherein at least a subset of the second nucleic acid strands are covalently attached to the first nucleic acid strand via an interstrand crosslink in the branch hybridization region; and b) a sequential series of probe panels, wherein each panel comprises multiple probe species, and wherein each probe species comprises (i) a barcode recognition sequence complementary to a different barcode sequence, and (ii) an adapter region, which is the same or different from the adapter region of a different probe species in the panel; wherein the adapter region is complementary to the adapter hybridization region of at least one of the species of bNA structures.

In some aspects, provided herein is a kit, comprising: a) at least two different species of branched DNA structures, wherein each species of branched DNA structure comprises: a first nucleic acid strand covalently connected to a plurality of second nucleic acid strands at a plurality of branch points, wherein each of the second nucleic acid strands is covalently linked to a plurality of detectable moieties at a plurality of triazole linkage points, and wherein the first nucleic acid strand comprises an adapter hybridization region; and b) a sequential series of probe panels, wherein each panel comprises multiple probe species, and wherein each probe species comprises (i) a barcode recognition sequence complementary to a different barcode sequence, and (ii) an adapter region, which is the same or different from the adapter region of a different probe species in the panel; wherein the adapter region is complementary to the adapter hybridization region of at least one of the species of bNA structures.

### BRIEF DESCRIPTION OF THE DRAWINGS

The drawings illustrate certain features and advantages of this disclosure. These embodiments are not intended to limit the scope of the appended claims in any manner.

FIG. 1A depicts exemplary probe designs for a branched DNA structure utilizing click-chemistry based covalent linkages between nucleic acid strands.

FIG. 1B depicts an exemplary step-wise assembly process for assembling a branched DNA structure utilizing click-chemistry based covalent linkages between nucleic acid strands.

FIG. 1C depicts an exemplary assembled branched DNA structure utilizing click-chemistry based covalent linkages between nucleic acid strands.

FIG. 2A depicts exemplary probe designs for a branched DNA structure utilizing cyanovinylcarbazole nucleoside analogue crosslinking between nucleic acid strands.

FIG. 2B depicts an exemplary UV irradiation step in order to form a fully assembled and crosslinked branched DNA structure utilizing a crosslinkable moiety between nucleic acid strands.

FIG. 2C depicts an exemplary step-wise assembly process for assembling a branched DNA structure utilizing crosslinkable moieties between nucleic acid strands.

FIG. 3 is an example workflow of analysis of a biological sample (e.g., a cell or tissue sample) using an opto-fluidic instrument, according to various embodiments.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

In some aspects, provided herein are branched nucleic acid (bNA) structures and methods of using the branched nucleic acid structures for detecting a region of interest in a target nucleic acid molecule. In some embodiments, the bNA structures are branched DNA structures (bDNA). In some embodiments, the bNA structures comprise interstrand crosslinks between branch hybridization regions (BHRs) in a first nucleic acid strand, and complementary reporter hybridization regions in detectably labeled probes. In some embodiments, the bNA structures comprise interstrand crosslinks between BHRs in a first nucleic acid strand and complementary hybridization regions (BHR's) in a second nucleic acid strand. In some embodiments, the bNA structures comprise interstrand crosslinks between reporter regions in second nucleic acid strands and complementary reporter regions in detectably labeled probes. In some embodiments, the covalent linkages are between a nucleic acid strand of the bNA structure and another nucleic acid strand or a detectable moiety. In some embodiments, the covalent linkages are formed using click chemistry.

In some aspects, the present application addresses various limitations of existing methods utilizing branched nucleic acid structures for signal amplification. Certain existing methods using bNA structures rely on hybridization between probes to maintain the bNA structure during detection. Such reliance on hybridization poses several problems. First, such large branched DNA structures require multiple independent hybridization events to generate the branched structure, resulting in low hybridization efficiency. The more hybridization events that need to take place to form the bNA structure, the lower the hybridization efficiency will be. Second, alterations to pH and salt concentrations can affect the stability of the hybridized structure. The present application addresses these and other problems. By covalently attaching (e.g., crosslinking) the individual nucleic acids within the branched structure, the entire complex is stabilized and is used for in situ hybridization to a target nucleic acid in a biological sample (e.g., a cell or tissue sample). In some aspects, the covalently linked bNA structure is formed before contacting the biological sample. Thus, in some aspects, the covalently linked (e.g., crosslinked) bNA structure only relies on a single hybridization event of the bNA structure to its target sequence (rather than numerous hybridization events between nucleic acid strands to form the bNA structure), greatly increasing the efficiency of the detected signal.

In some aspects, the present application provides nucleic acid strand designs and methods for formation of crosslinked branched nucleic acid (bNA) structures with improved properties for in situ detection of target nucleic acid molecules and/or target analytes in a biological sample. In some embodiments, provided herein are bNA structures assembled from a first nucleic acid strand comprising an adapter hybridization region and a plurality of branch hybridization regions (BHRs), a second nucleic acid strand comprising a complementary branch hybridization region (BHR′) and a detectable label or an overhang region comprising one or more reporter regions (e.g., for detection by sequence or for directly or indirectly binding to one or more detectably labeled probes), wherein at least a subset of the second nucleic acid strands are covalently attached to the first nucleic acid strand.

In some aspects, the present application provides crosslinked bNA structures that hybridize to a target nucleic acid and contain a plurality of detectable labels, improving the number of signals generated per hybridization event. Without being bound by theory, in some aspects the hybridization efficiency of a branched structure, such as in the present application, hybridizing to a target nucleic acid decreases as more hybridization events are designed to occur. By crosslinking the branched structure via interstrand crosslinks prior to contacting a biological sample, the hybridization efficiency of the bNA with the target nucleic acid is greatly improved. Moreover, utilizing crosslinks between the nucleic acid strands in the bNA structure allows for shorter nucleic acid strand designs, thereby reducing the costs and improving the fidelity of nucleic acid strand synthesis.

In some embodiments, the detectable label is a fluorophore having an excitation peak between 480 nm and 500 nm. In some embodiments, the detectable label is a fluorophore having an excitation peak between 520 nm and 540 nm. In some embodiments, the detectable label is a fluorophore having an excitation peak between 590 nm and 600 nm. In some embodiments, the detectable label is a fluorophore having an excitation peak between 640 nm and 660 nm.

### II. Branched Nucleic Acid Structures and Methods

In some embodiments, provided herein is a method for analyzing a biological sample, comprising: (a) contacting the biological sample with a branched nucleic acid (bNA) structure, wherein the bNA structure hybridizes to an adapter region in a target nucleic acid or in a probe or probe set hybridized to a target nucleic acid in the biological sample. The bNA structure can be any of the bNA structures described herein. In some embodiments, the adapter region is a barcode sequence or subunit thereof. In some embodiments, the adapter region is in a probe comprising a recognition sequence that hybridizes to a target nucleic acid in the biological sample. In some embodiments, the adapter region is a marker sequence in a nucleic acid analyte, and the bNA structure hybridizes directly to the target analyte. In some embodiments, the adapter region is a barcode sequence or subunit thereof in a probe, amplification product (e.g., rolling circle amplification product) or labeling agent that is associated with an analyte in the biological sample. Various analytes, probes, and labeling agents are described in Section III below.

In some instances, a branched nucleic acid structure as described herein is an amplification structure that provides multiple replicate signal generation sites on an individual nucleic acid molecule. In some embodiments, two or more second nucleic acid “branches” are connected to the first nucleic acid strand (e.g., via a covalent linkage or hybridization). In some embodiments, the assembled bNA structure comprises at least two second nucleic acid strands covalently attached to a single first nucleic acid strand, thereby forming at least two branches from a single first nucleic acid strand. In some embodiments, the assembled bNA structure comprises at least two second nucleic acid strands hybridized to a single first nucleic acid strand, thereby forming at least two branches from a single first nucleic acid strand. In some embodiments, the bNA structure is a DNA bNA structure.

In some aspects, provided herein is a bNA structure comprising a plurality of nucleic acid strands, wherein each of the nucleic acid strands is covalently linked to a plurality of detectable moieties at a plurality of triazole linkage points. In some embodiments, the nucleic acid strands are second nucleic acid strands, which are hybridized to, covalently attached to, and/or crosslinked to first nucleic acid strands in the bNA structure. In some embodiments, provided herein is a bNA structure comprising a first nucleic acid strand covalently connected to a plurality of second nucleic acid strands at a plurality of branch points, wherein each of the second nucleic acid strands is covalently linked to a plurality of detectable moieties at a plurality of triazole linkage points. In some embodiments, the branch points comprise interstrand crosslinks between the second nucleic acid strand and the first nucleic acid strand. In some embodiments, the branch points comprise triazole linkages.

In embodiments, provided herein is a bNA structure comprising a first nucleic acid strand covalently connected to a plurality of second nucleic acid strands at a plurality of branch points, wherein each of branch point covalent attachments is a triazole linkage. In embodiments, the bNA structure comprises at least three separate branch points on a single first nucleic acid strand. In embodiments, the bNA structure comprises at least a single first nucleic acid strand that comprises at least three separate branch points. In some embodiments, each of the second nucleic acid strands is covalently attached to a plurality of detectable moieties at a plurality of linkage points. In some embodiments, the linkage points comprise triazole linkages. In some embodiments, the linkage points comprise thiol linkages.

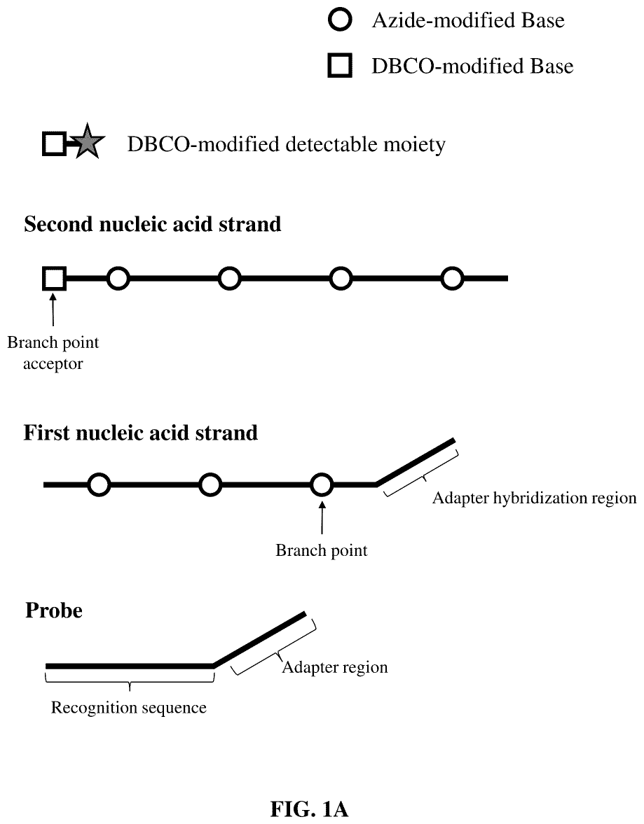

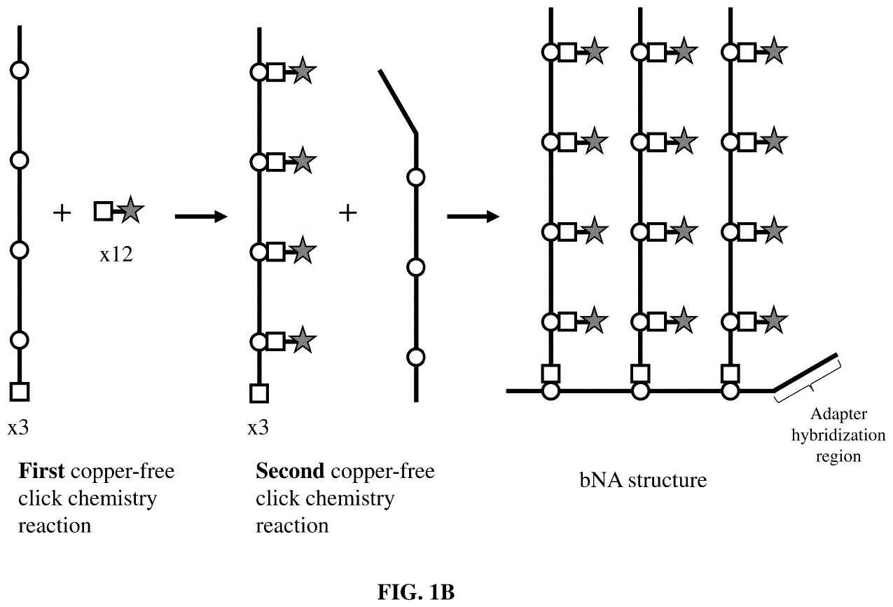

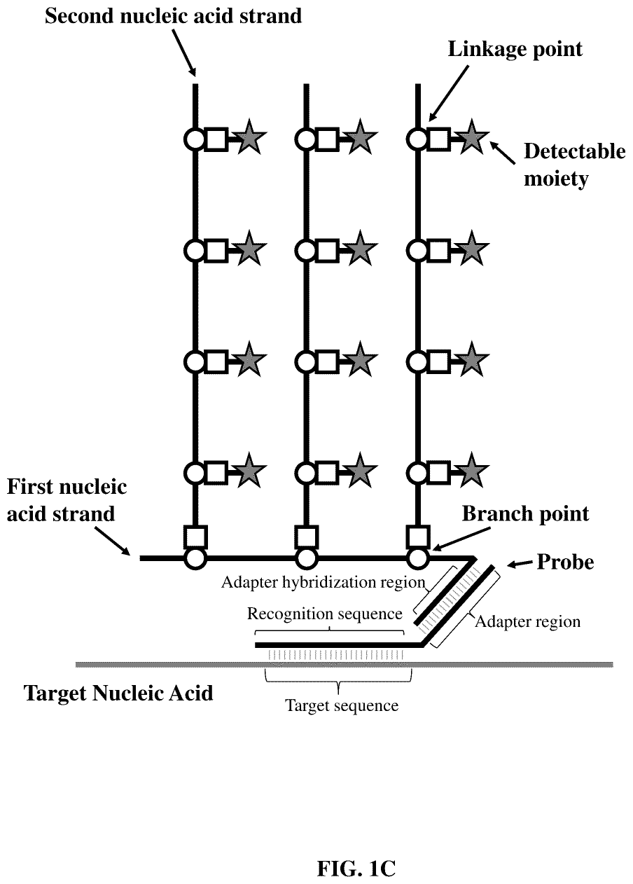

An exemplary set of nucleic acids and a modified detectable moiety for assembling a bNA structure according to the present disclosure is illustrated in FIG. 1A. As shown in FIG. 1A, in some aspects provided herein is a first nucleic acid strand comprising a plurality of branch points, a second nucleic acid strand comprising a branch point acceptor and a plurality of acceptor moieties, and a detectable moiety modified with a moiety for covalent attachment to the acceptor moieties. For example, in some embodiments the detectable moiety is a dibenzocyclooctyne (DBCO)-modified detectable moiety, and the acceptor moiety is an azide modified base (as shown in FIG. 1A). In another example (not illustrated), the detectable moiety is be an azide-modified detectable moiety, and the acceptor moiety is a DBCO or alkyne.

In some embodiments, the detectable moiety is attached to the acceptor moiety via a click chemistry reaction. In some embodiments, the click chemistry reaction is a strain promoted alkyne-azide cycloaddition (SPAAC). In some embodiments, the click chemistry reaction is run in aqueous buffer. In some embodiments, the click chemistry reaction is run in organic solvent.

In some embodiments, the detectable moiety is or comprises a thiol-reactive moiety. In some embodiments, the acceptor moiety is a thiol moiety. In some embodiments, the second nucleic acid strand comprises a plurality of internal thiol modifier groups for attachment of the thiol-reactive detectable moiety. A deoxythymidine (dT) modified with a spacer, and a thiol group can be used for adding internal thiol modifier groups at desired locations within oligonucleotide sequences. In some embodiments, this thiol modifier reacts directly with maleimides or haloacetamides. The structure of an exemplary internal thiol modifier dT is provided below (S-Bz-Thiol-Modifier C6-dT: 5′-(4,4′-Dimethoxytrityl)-5-[N-(6-(3-benzoylthiopropanoyl)-aminohexyl)-3-acrylamido]-2′-deoxyuridine, 3′-[(2-cyanoethyl)-(N,N-diisopropyl)]-phosphoramidite):

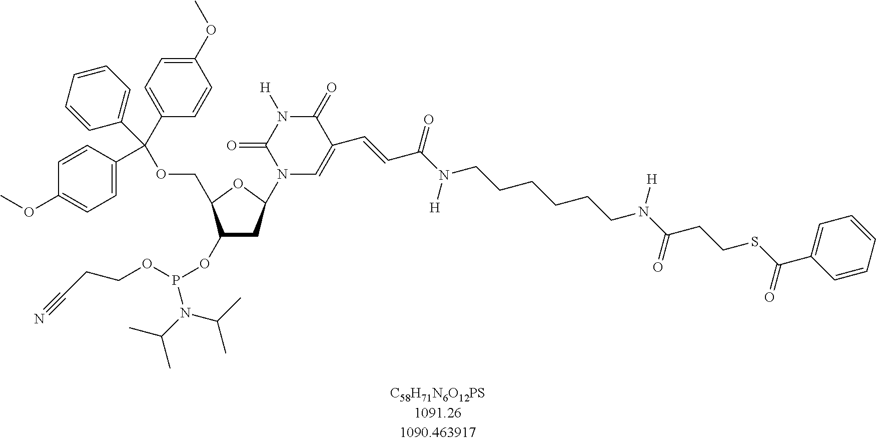 [SMILES: COc1ccc(C(OC[C@H]2O[C@@H](n3cc(/C=C/C(=O)NCCCCCCNC(=O)CCSC(=O)c4ccccc4)c(=O)[nH]c3=O)C[C@H]2OP(OCCC#N)N(C(C)C)C(C)C)(c2ccccc2)c2ccc(OC)cc2)cc1]

In some embodiments, the branch point acceptor is a thiol-reactive moiety. For example, in some embodiments the branch point acceptor is a maleimide moiety, such as a 5′ maleimide amidite modification. In some embodiments, the branch point acceptor is a maleimide NHS oligo modification. In some embodiments, the branch point acceptor is at the 5′ end of the second nucleic acid strand. In some embodiments, the branch point acceptor is at the 3′ end of the second nucleic acid strand. In some embodiments, the branch point acceptor is internal to the second nucleic acid strand, optionally wherein the second nucleic acid strand comprises a sequence that hybridizes to the first nucleic acid strand. Thiol-modified oligonucleotides are described, for example, in U.S. Pat. No. 11,390,643, the content of which is herein incorporated by reference in its entirety.

In some embodiments, the detectable moiety does not comprise a moiety that reacts with the branch point moieties on the first nucleic acid strand or the branch point acceptor, but does reach with the acceptor moieties in the second nucleic acid strand. In some embodiments, orthogonal chemistries are used for attachment of the detectable moieties to the second nucleic acid strands and attachment of the second nucleic acid strands to the first nucleic acid strands. In some embodiments, the attachment of the detectable moieties to the second nucleic acid strand and attachment of the second nucleic acid strands to the first nucleic acid strand is performed sequentially.

In some embodiments, provided herein is a method of making a bNA structure. In some embodiments, the method comprises using click chemistry to “click” the nucleic acid strands of the bNA structure together in solution. In some embodiments, the resulting assembled bNA structure is contacted with a biological sample and allowed to bind directly or indirectly to a target nucleic acid. Thus, binding of the bNA to a probe or target nucleic acid in the biological sample can require only a single hybridization event. In some aspects, this greatly increases the efficiency of signal buildup from the detectably labeled bNA structure, compared to a bNA structure requiring many probe hybridization. In an example illustrated in FIG. 1B, three second nucleic acid strands comprising four acceptor moieties (e.g., azide-modified nucleotides) each are added together with DBCO-modified detectable moieties (e.g., DBCO-modified fluorophores) to start a copper-free click reaction to attach the detectable moieties (e.g., fluorophores) to the second nucleic acid strands. In some embodiments, the second nucleic acid strand comprises 4, 5, 6, 7, 8, 9, 10, or more acceptor moieties. As shown in FIG. 1B, after performing the first copper-free click chemistry reaction to attach the detectable moieties, the resulting second nucleic acid strand/detectable moiety complexes are then added to the first nucleic acid strand. The second nucleic acid strands comprise a branch point acceptor, and the first nucleic acid strand comprises branch point moieties. In the example shown, the first nucleic acid strand contains 3 branch point moieties (e.g., azide-modified nucleotides). In some embodiments, the first nucleic acid strand comprises 4, 5, 6, 7, 8, 9, 10, or more branch point moieties. A second copper-free click chemistry reaction is then initiated, as shown in FIG. 1B, resulting in a bNA complex. In the example illustrated in FIG. 1B, the series of two reactions results in a bNA complex comprising 12 detectable moieties (e.g., 12 fluorophores) and 3 second nucleic acid strands conjugated to a first nucleic acid strand. In some embodiments, the second nucleic acid strands are “L-shaped” probes, comprising a hybridization region complementary to a hybridization region in the first nucleic acid strand, and an overhang region comprising the acceptor moieties. In some embodiments, the second nucleic acid strands to not comprise a hybridization region complementary to a sequence in the first nucleic acid strand.

In some aspects, provided herein is a method for analyzing a biological sample, comprising: (a) contacting the biological sample with a branched nucleic acid (bNA) structure comprising a first nucleic acid strand covalently connected to a plurality of second nucleic acid strands at a plurality of branch points, wherein each of the second nucleic acid strands is covalently linked to a plurality of detectable moieties at a plurality of triazole linkage points, wherein the first nucleic acid strand comprises an adapter hybridization region that hybridizes to an adapter region in a probe or probe set hybridized to a target nucleic acid in the biological sample; and detecting the bNA structure at a location in the biological sample, thereby the target nucleic acid at the location in the biological sample. An exemplary complex comprising the bNA hybridized to a probe, wherein the probe is hybridized to a target sequence in a target nucleic acid is illustrated in FIG. 1C. In some embodiments, the target nucleic acid is any target nucleic acid described herein (e.g., in Section III). In some aspects, after imaging the biological sample to detect the bNA structure, the method comprises removing the bNA structure. In some embodiments, after imaging the biological sample to detect the bNA structure, the method comprises cleaving the bNA structure and/or cleaving the detectable moieties from the bNA structure. In some embodiments, the bNA structure is cleaved at one or more linkage points or branch points.

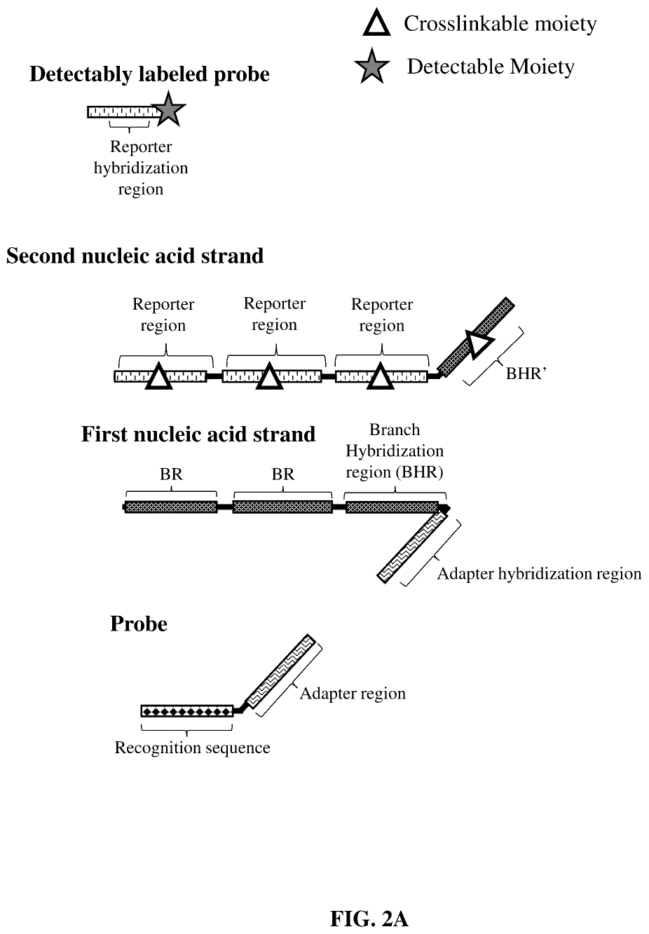

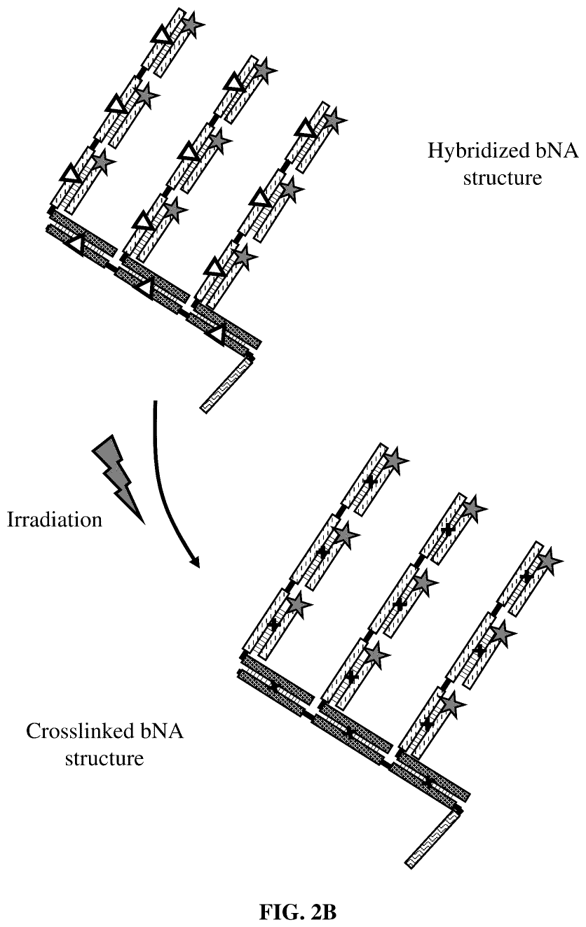

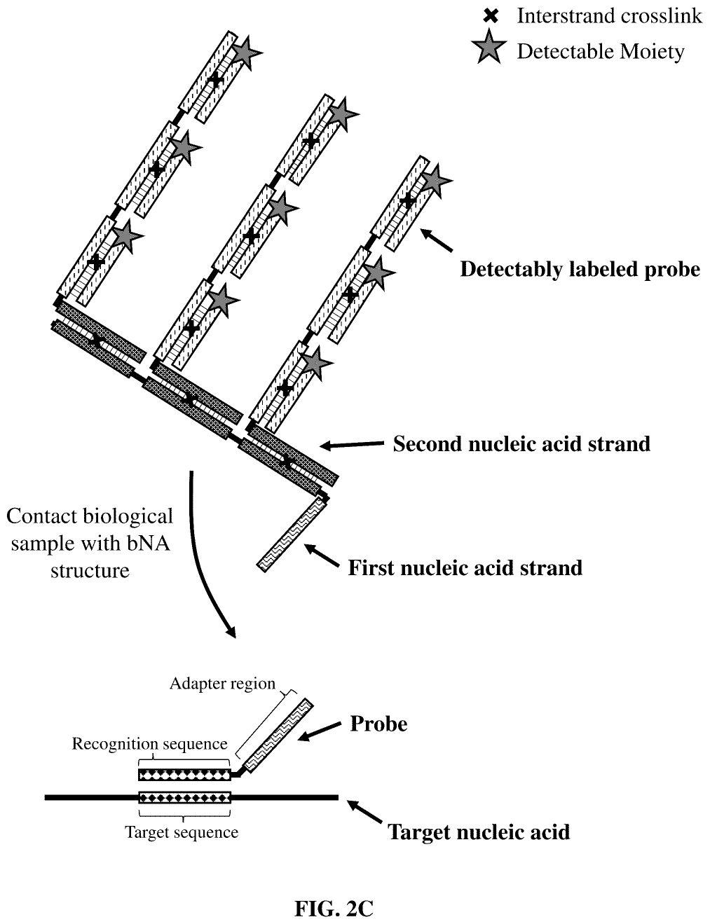

In some aspects, provided herein are bNA structures and methods of producing bNA structures comprising covalent interstrand linkages between first nucleic acid strands and second nucleic acid strands. An exemplary set of nucleic acids for assembling a bNA structure comprising covalent interstrand linkages (e.g., interstrand crosslinks) according to the present disclosure is illustrated in FIG. 2A. In some embodiments, the first nucleic acid strand comprises a plurality of branch hybridization regions (BHRs), and the second nucleic acid strand comprises a complementary branch hybridization region (BHR′). BHR′ is complementary to BHR. In some embodiments, as illustrated in FIG. 2A, the second nucleic acid strand comprises a crosslinkable moiety within BHR′. Additionally or alternatively, the first nucleic acid strand could comprise a crosslinkable moiety within BHR. The second nucleic acid strand comprises a plurality of reporter regions. In some embodiments, the method comprises detecting the bNA structure by detecting the reporter region(s) in the bNA structure. Examples of methods for detecting reporter regions are described in detail in Section II.E. In some embodiments, the reporter region binds directly or indirectly to a reporter hybridization region in a detectably labeled probe. In some embodiments, the reporter region is complementary to the reporter hybridization region, and the second nucleic acid strand hybridizes to a plurality of detectable labeler probes. In other embodiments, the reporter region hybridizes to additional nucleic acid strands for another layer of branching (e.g., third nucleic acid strands are hybridized to the second nucleic acid strand, wherein each third nucleic acid strand hybridizes to a plurality of detectably labeled probes). In some embodiments, the reporter region is analyzed by nucleic acid sequencing in the biological sample.

In some embodiments, the crosslinkable moiety is any of those described in Section II.B below. In other embodiments, a crosslinkable intercalating agent such as a psoralen is contacted with the bNA structure after hybridization of the first nucleic acid strands, second nucleic acid strands, and detectably labeled probes.

As illustrated in FIG. 2B, in some embodiments, the crosslinked bNA structure is generated by irradiating the hybridized bNA structure comprising photocrosslinkable moieties (or a crosslinkable intercalating agent) to generate interstrand crosslinks: (a) between the detectably labeled probes and the second nucleic acid strands, (b) between the second nucleic acid strands and the first nucleic acid strands, or (c) between the detectably labeled probes and the second nucleic acid strands and between the second nucleic acid strands and the first nucleic acid strands.

In some embodiments, a crosslinkable moiety (e.g., 5-Bromodeoxy-Uracil (BrdU)) is incorporated into the BHR′ part and into the reporter regions of each second nucleic acid strand. The second nucleic acid strands can then be crosslinked to the first nucleic acid strands and to the detectably labeled probes, as shown in FIGS. 2A-2B. In some embodiments, the crosslinking is performed in solution, before contacting the biological sample with the bNA structure, as illustrated in FIG. 2C. In some embodiments, the nucleic acid strands and detectably labeled probes are contacted with the biological sample, allowed to hybridize in the biological sample, and then crosslinked.

An exemplary method for analyzing a biological sample is illustrated in FIG. 2C, comprising: (a) contacting the biological sample with a crosslinked branched nucleic acid (bNA) structure comprising a first nucleic acid strand and a plurality of second nucleic acid strands connected to each other by a plurality of interstrand crosslinks, wherein the second nucleic acid strands are connected to a plurality of detectably labeled probes via a plurality of interstrand crosslinks, wherein the first nucleic acid strand of the bNA hybridizes to a probe that binds to a target nucleic acid in the biological sample and (b) detecting the hybridized bNA structure at a location in the biological sample, thereby detecting the target nucleic acid at the location in the biological sample. In some embodiments, the target nucleic acid is any target nucleic acid described herein (e.g., in Section III). In some aspects, after imaging the biological sample to detect the bNA structure, the method comprises removing the bNA structure. In some embodiments, after imaging the biological sample to detect the bNA structure, the method comprises reversing the interstrand crosslinks (e.g., as described in Section II.D below).

In some aspects provided herein is a method for analyzing a biological sample, comprising: (a) contacting the biological sample with a crosslinked bNA structure comprising a first nucleic acid strand and a plurality of second nucleic acid strands, wherein the first nucleic acid strand comprises: (i) an adapter hybridization region that hybridizes to an adapter region in a target nucleic acid or in a probe or probe set hybridized to a target nucleic acid in the biological sample, and (ii) a plurality of branch hybridization regions (BHRs), wherein each of the second nucleic acid strands comprises a complementary branch hybridization region (BHR′) and a detectable label or an overhang region comprising one or more reporter regions for directly or indirectly binding to one or more detectably labeled probes, wherein BHR′ hybridizes to BHR, wherein at least a subset of the second nucleic acid strands are covalently attached to the first nucleic acid strand via an interstrand crosslink in the BHR, and (b) detecting the hybridized bNA structure at a location in the biological sample, thereby detecting the target nucleic acid at the location in the biological sample.

In some embodiments, provided herein is a method for analyzing a biological sample, comprising: (a) contacting the biological sample with a bNA structure comprising a first nucleic acid strand covalently connected to a plurality of second nucleic acid strands at a plurality of branch points, wherein each of the second nucleic acid strands is covalently linked to a plurality of detectable moieties at a plurality of triazole linkage points, wherein the first nucleic acid strand comprises an adapter hybridization region that hybridizes to an adapter region in a target nucleic acid or in a probe or probe set hybridized to a target nucleic acid in the biological sample; and (b) detecting the plurality of hybridized bNA structures at a location in the biological sample, thereby detecting the plurality of target nucleic acids at the location in the biological sample.

### A. Nucleic Acid Strands

In some embodiments, the methods provided herein comprise contacting a biological sample with a plurality of nucleic acid strands capable of forming a branched nucleic acid (bNA) structure, and crosslinking the nucleic acid strands to form a crosslinked bNA structure in the biological sample. The crosslinked bNA structure bound to a target nucleic acid in the biological sample can then be detected. In some embodiments, the methods provided herein comprise contacting a biological sample with branched nucleic acid (bNA) structures and detecting complexes formed between the bNA structures and target nucleic acids or probe or probe sets hybridized to a target nucleic acid in the biological sample. Such bNA structures and resulting hybridization complexes are described in further detail below.

### (i) First Nucleic Acid Strands

In some aspects, provided herein are first nucleic acid strands. A first nucleic acid strand comprises an adapter hybridization region and at least one branch hybridization region (BHR). In some embodiments, the adapter hybridization region is a sequence complementary to an adapter region in a target nucleic acid or in a probe or probe set hybridized to a target nucleic acid in the biological sample. In some embodiments, the adapter hybridization region is a sequence complementary to an adapter region in a target nucleic acid in the biological sample. In some embodiments, the adapter hybridization region is a sequence complementary to a probe or probe set hybridized to a target nucleic acid in the biological sample. In some embodiments, a target nucleic acid is a nucleic acid analyte, a nucleic acid product such as a cDNA or rolling circle amplification product, or an oligonucleotide reporter associated with a non-nucleic acid analyte. In some embodiments, the adapter hybridization region is about 15 and about 50 nucleotides in length, between about 16 and about 50 nucleotides in length, between about 20 and about 50 nucleotides in length, between about 20 and about 40 nucleotides in length, between about 16 and about 40 nucleotides in length, between about 16 and about 30 nucleotides in length.

In some embodiments, the adapter region is in a probe or probe set, and wherein the method comprises contacting the biological sample with the probe or probe set, wherein the probe or probe set comprises (i) a recognition sequence that hybridizes to the target nucleic acid in the biological sample, and (ii) an overhang region comprising the adapter region.

In some embodiments, the BHR is a sequence complementary to the BHR′ on the second nucleic acid strands. In some embodiments, the BHR is about 15 and about 50 nucleotides in length, between about 16 and about 50 nucleotides in length, between about 20 and about 50 nucleotides in length, between about 20 and about 40 nucleotides in length, between about 16 and about 40 nucleotides in length, between about 16 and about 30 nucleotides in length.

In some embodiments, the first nucleic acid strand comprises 2, 3, 4, 5, 6, or more BHRs. In some embodiments, the first nucleic acid comprises between 2 and 20 BHRs. In some embodiments, the first nucleic acid comprises between 2 and 4, between 2 and 5, or between 2 and 6 BHRs. In some embodiments, the first nucleic acid strand comprises 3 BHRs. In some embodiments, the first nucleic acid strand comprises 4 BHRs. In some embodiments, the at least two BHRs are separated by a linker of at least 5 nm in length. In some embodiments, the at least two BHRs are separated by a spacer region of at least 4 nucleotides in length. In some embodiments, the spacer region is at least 5, at least 6, at least 7, or at least 8 nucleotides in length. In some embodiments, the spacer region is no more than 10, no more than 9, no more than 8, or no more than 7 nucleotides in length. In some embodiments, the spacer region is between 4 and 10, between 4 and 9, between 4 and 8, or between 5 and 8 nucleotides in length. In some embodiments, the spacer region is between 4 and 20, between 4 and 15, between 4 and 10, between 8 and 20, or between 8 and 15 nucleotides in length. In some embodiments, each of the spacer regions has the same sequence. In some embodiments, the spacer regions have different sequences. In some embodiments, the spacer regions have random sequences. In some embodiments, the spacer region sequences are a sequence of adenines and/or thymidines. In some embodiments, each of the BHRs has the same sequence (e.g., the BHRs are copies of the same sequence to allow hybridization of multiple molecules of the same second nucleic acid strand to the first nucleic acid strand).

In some embodiments, the first nucleic acid strand comprises, from 5′ to 3′ or from 3′ to 5′, an adapter hybridization region, a spacer region, and a first BHR, a spacer region, and a second BHR. In some embodiments, the first nucleic acid strand comprises, from 5′ to 3′ or from 3′ to 5′, an adapter hybridization region, a spacer region, and a first BHR, a spacer region, and a second BHR, a spacer region, a third BHR, a spacer region, and a fourth BHR. In some embodiments, the first, second, third, and fourth BHRs are individually between 15 and 25 nucleotides in length, between 16 and 25 nucleotides in length, between 16 and 22 nucleotides in length, or between 16 and 20 nucleotides in length. In some embodiments, the BHRs are individually about 16 nucleotides in length. FIG. 2A illustrates an exemplary first nucleic acid strand comprising an adapter hybridization region and three BHRs.

In some embodiments, the first nucleic acid strand comprises an adapter hybridization region and at least one branch points. In some embodiments the branch points comprise nucleotides containing crosslinkable moieties. In some embodiments, the branch points comprise nucleotides containing crosslinkable moieties amenable to click reaction chemistry. In some embodiments, first nucleic acid strands comprising branch points do not contain sequence complementarity with the second nucleic acid strands. In some embodiments, the branch points are within BHRs. In some embodiments, the first nucleic acid strand comprises 2, 3, 4, 5, 6, or more branch points. In some embodiments, the first nucleic acid comprises between 2 and 20 branch points. In some embodiments, the first nucleic acid comprises between 2 and 4, between 2 and 5, or between 2 and 6 branch points. In some embodiments, the first nucleic acid strand comprises 3 branch points. In some embodiments, the first nucleic acid strand comprises 4 branch points. In some embodiments, each of the branch points is separated by a linker of at least 5 nm in length. In some embodiments, the at least two branch points are separated by a spacer region of at least 4 nucleotides in length. In some embodiments, the spacer region is at least 5, at least 6, at least 7, or at least 8 nucleotides in length. In some embodiments, the spacer region is no more than 10, no more than 9, no more than 8, or no more than 7 nucleotides in length. In some embodiments, the spacer region is between 4 and 10, between 4 and 9, between 4 and 8, or between 5 and 8 nucleotides in length. In some embodiments, the spacer region is between 4 and 20, between 4 and 15, between 4 and 10, between 8 and 20, or between 8 and 15 nucleotides in length. In some embodiments, each of the spacer regions has the same sequence. In some embodiments, the spacer regions have different sequences. In some embodiments, the spacer regions have random sequences. In some embodiments, the spacer region sequences are a sequence of adenines and/or thymidines.

In some embodiments, the first nucleic acid strand comprises, from 5′ to 3′ or from 3′ to 5′, an adapter hybridization region, a spacer region, and a first branch point, a spacer region, and a second branch point. In some embodiments, the first nucleic acid strand comprises, from 5′ to 3′ or from 3′ to 5′, an adapter hybridization region, a spacer region, and a first branch point, a spacer region, and a second branch point, a spacer region, a third branch point, a spacer region, and a fourth branch point. FIG. 1A illustrates an exemplary first nucleic acid strand comprising an adapter hybridization region and three branch points.

In some embodiments, the first nucleic acid strand is between about 40 and 500 nucleotides in length. In some embodiments, the first nucleic acid strand is between about 50 and about 500 nucleotides in length. In some embodiments, the first nucleic acid strand is between any of 40 and about 60, about 45 and about 60, about 50 and about 60, or about 45 and about 55 nucleotides in length.

### (ii) Second Nucleic Acid Strands

In some embodiments, also provided herein are second nucleic acid strands. In some cases the second nucleic acid strand comprises a complementary branch hybridization region (BHR′) and a detectable label or an overhang region comprising one or more reporter regions for directly or indirectly binding to one or more detectably labeled probes. In some embodiments, the second nucleic acid strand comprises a BHR′ and a detectable label. In some embodiments, the second nucleic acid strand comprises a BHR′ and an overhang region comprising one or more reporter regions for directly or indirectly binding to one or more detectably labeled probes.

In some embodiments, the BHR′ is a sequence complementary to the BHR sequence on the first nucleic acid strand. In some embodiments, the BHR′ is about 15 and about 50 nucleotides in length, between about 16 and about 50 nucleotides in length, between about 20 and about 50 nucleotides in length, between about 20 and about 40 nucleotides in length, between about 16 and about 40 nucleotides in length, between about 16 and about 30 nucleotides in length. In some embodiments, the BHR′ hybridizes to the BHR on the first nucleic acid, wherein at least a subset of the second nucleic acid strands are covalently attached to the first nucleic acid strand via an interstrand crosslink between the BHR′ and BHR.

In some embodiments, the detectable label is a label that can be measured and quantitated. The detectable label can comprise a directly or indirectly detectable moiety, e.g., any fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. Further description of the detectable label is found below.

In some embodiments, the second nucleic acid strands comprises an overhang region comprising a plurality of reporter regions, wherein the bNA structure comprises a plurality of detectably labeled probes hybridized to the reporter regions via complementary reporter hybridization regions, optionally wherein at least a subset of the reporter regions are covalently attached to the hybridized detectably labeled probes via an interstrand crosslink.

In some embodiments, the second nucleic acid strand comprises 2, 3, 4, 5, 6, or more reporter regions. In some embodiments, the second nucleic acid comprises between 2 and 20 reporter regions. In some embodiments, the second nucleic acid comprises between 2 and 4, between 2 and 5, or between 2 and 6 reporter regions. In some embodiments, the second nucleic acid strand comprises 3 reporter regions. In some embodiments, the second nucleic acid strand comprises 4 reporter regions. In some embodiments, the at least two reporter regions are separated by a linker of at least 5 nm in length. In some embodiments, the at least two reporter regions are separated by a spacer region of at least 4 nucleotides in length. In some embodiments, the spacer region is at least 5, at least 6, at least 7, or at least 8 nucleotides in length. In some embodiments, the spacer region is no more than 10, no more than 9, no more than 8, or no more than 7 nucleotides in length. In some embodiments, the spacer region is between 4 and 10, between 4 and 9, between 4 and 8, or between 5 and 8 nucleotides in length. In some embodiments, the spacer region is between 4 and 20, between 4 and 15, between 4 and 10, between 8 and 20, or between 8 and 15 nucleotides in length. In some embodiments, each of the spacer regions has the same sequence. In some embodiments, the spacer regions have different sequences. In some embodiments, the spacer regions have random sequences. In some embodiments, the spacer region sequences are a sequence of adenines and/or thymidines. In some embodiments, each of the reporter regions has the same sequence (e.g., the reporter regions are copies of the same sequence to allow hybridization of multiple molecules of the same detectably labeled probes to the second nucleic acid strand).

In some embodiments, the second nucleic acid strand comprises, from 5′ to 3′ or from 3′ to 5′, a BHR′, a spacer region, and a first reporter region, a spacer region, and a second reporter region. In some embodiments, the first nucleic acid strand comprises, from 5′ to 3′ or from 3′ to 5′, a BHR′, a spacer region, and a first reporter region, a spacer region, and a second reporter region, a spacer region, a third reporter region, a spacer region, and a fourth reporter region. In some embodiments, the first, second, third, and fourth reporter region are individually between 15 and 25 nucleotides in length, between 16 and 25 nucleotides in length, between 16 and 22 nucleotides in length, or between 16 and 20 nucleotides in length. In some embodiments, the BHRs are individually about 16 nucleotides in length. FIG. 2A illustrates an exemplary second nucleic acid strand comprising a BHR′ and three reporter regions.

In some embodiments, the second nucleic acid strand comprises branch point acceptor and a detectable label or an overhang region comprising one or more branch points. In some embodiments, the second nucleic acid strand comprises a branch point acceptor and a detectable label. In some embodiments, the second nucleic acid strand comprises a branch point acceptor and an overhang region comprising one or more branch points.

In some embodiments, the branch point acceptor is a modified crosslinkable nucleotide comprising a moiety that can exclusively react with a branch point modified nucleotide within the first nucleic acid strand. In some embodiments, the branch point acceptor is a modified crosslinkable nucleotide comprising a moiety that can exclusively react with a branch point modified nucleotide within the first nucleic acid strand, wherein the reaction comprises click reaction chemistry. In some embodiments the reaction between the branch point acceptor and the branch point results in a covalent crosslink between the first nucleic acid strand and the second nucleic acid strand.

In some embodiments the branch points within the second nucleic acid strand comprise nucleotides containing crosslinkable moieties. In some embodiments, the branch points comprise nucleotides containing crosslinkable moieties amenable to click reaction chemistry. In some embodiments, second nucleic acid strands comprising branch points do not contain sequence complementarity with the detectably labeled probe. In some embodiments, the branch points are within reporter regions containing sequence complementarity with the detectably labeled probe. In some embodiments, the second nucleic acid strand comprises 2, 3, 4, 5, 6, or more branch points. In some embodiments, the second nucleic acid comprises between 2 and 20 branch points. In some embodiments, the second nucleic acid comprises between 2 and 4, between 2 and 5, or between 2 and 6 branch points. In some embodiments, the second nucleic acid strand comprises 3 branch points. In some embodiments, the second nucleic acid strand comprises 4 branch points. In some embodiments, the at least two branch points are separated by a spacer region of at least 4 nucleotides in length. In some embodiments, the spacer region is at least 5, at least 6, at least 7, or at least 8 nucleotides in length. In some embodiments, the spacer region is no more than 10, no more than 9, no more than 8, or no more than 7 nucleotides in length. In some embodiments, the spacer region is between 4 and 10, between 4 and 9, between 4 and 8, or between 5 and 8 nucleotides in length. In some embodiments, the spacer region is between 4 and 20, between 4 and 15, between 4 and 10, between 8 and 20, or between 8 and 15 nucleotides in length. In some embodiments, each of the spacer regions has the same sequence. In some embodiments, the spacer regions have different sequences. In some embodiments, the spacer regions have random sequences. In some embodiments, the spacer region sequences are a sequence of adenines and/or thymidines.

In some embodiments, the second nucleic acid strand comprises, from 5′ to 3′ or from 3′ to 5′, a BHR′, a spacer region, and a first branch point, a spacer region, and a second branch point. In some embodiments, the second nucleic acid strand comprises, from 5′ to 3′ or from 3′ to 5′, a BHR′, a spacer region, and a first branch point, a spacer region, and a second branch point, a spacer region, a third branch point, a spacer region, and a fourth branch point. FIG. 1A illustrates an exemplary second nucleic acid strand comprising a BHR′ and four branch points.

In some embodiments, the second nucleic acid strand is between about 40 and 500 nucleotides in length. In some embodiments, the second nucleic acid strand is between about 50 and about 500 nucleotides in length. In some embodiments, the second nucleic acid strand is between any of 40 and about 60, about 45 and about 60, about 50 and about 60, or about 45 and about 55 nucleotides in length.

(iii) Additional Nucleic Acid Strands for Branching

In some embodiments, any of the bNA structures described herein may comprise one or more additional levels of branching. For example, the second nucleic acid strands can hybridize or be covalently attached to third nucleic acid strands, which in turn can hybridize to detectably labeled probes or be covalently attached to detectably labeled moieties according to any of the methods disclosed herein.

### (iv) Detectably Labelled Probes

In some aspects, the methods provided herein comprise crosslinking detectably labelled probes to the second nucleic acid strands. In some embodiments, a detectably labeled probe herein comprises a reporter hybridization region and a detectable label. In some embodiments, the detectably labeled probes are individually between 15 and 25 nucleotides in length, between 15 and 30 nucleotides in length, between 15 and 40 nucleotides in length, or between 20 and 40 nucleotides in length. In some embodiments the reporter hybridization region contains complementarity with the reporter region in the second nucleic acid strands. In some embodiments, the reporter hybridization region is between 15 and 25 nucleotides in length, between 15 and 20 nucleotides in length, between 16 and 25 nucleotides in length, between 16 and 22 nucleotides in length, or between 20 and 25 nucleotides in length. In some embodiments, the reporter hybridization region is no more than any one of 16, 17, 18, 19, 20, 22, or 25 nucleotides in length.

In some embodiments, each of the second nucleic acid strands are covalently linked to a plurality of detectable moieties at a plurality of linkage points. In some embodiments, linkage points consist of a crosslinkable moiety within the reporter regions of the second nucleic acid strands, or branch points within the second nucleic acid strands.

In some embodiments, the detectably labeled probe herein comprises a branch point acceptor and a detectable label. In some embodiments, the branch point acceptor is a modified crosslinkable nucleotide comprising a moiety that can exclusively react with a branch point modified nucleotide within the second nucleic acid strand. In some embodiments, the branch point acceptor is a modified crosslinkable nucleotide comprising a moiety that can exclusively react with a branch point modified nucleotide within the second nucleic acid strand, wherein the reaction comprises click reaction chemistry. In some embodiments the reaction between the branch point acceptor and the branch point results in a covalent crosslink between the second nucleic acid strand and the detectably labeled probe. In some embodiments the detectably labeled probes comprising a branch point acceptor and a detectable label are individually between 5 and 25 nucleotides in length, between 5 and 30 nucleotides in length, between 5 and 40 nucleotides in length, or between 2 and 40 nucleotides in length. In some embodiments, the reporter region is between 10 and 25 nucleotides in length, between 10 and 20 nucleotides in length, between 12 and 25 nucleotides in length, between 16 and 22 nucleotides in length, or between 20 and 25 nucleotides in length. In some embodiments, the reporter region is no more than any one of 12, 14, 16, 18, 20, 22, or 25 nucleotides in length.

In some embodiments, the detectable label is a label that can be measured and quantitated. The detectable label can comprise a directly or indirectly detectable moiety, e.g., any fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

In some embodiments, the detectable label is a fluorophore. A fluorophore can comprise a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urcase.

Fluorescence detection in tissue samples can often be hindered by the presence of strong background fluorescence. Background fluorescence can include autofluorescence (that can arise from a variety of sources, including aldehyde fixation, extracellular matrix components, red blood cells, lipofuscin, and the like), as opposed to the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (Max Vision Biosciences), and/or a very intense black dye (e.g., Sudan Black, or comparable dark chromophore).

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycocrythrin.

Examples of bioluminescent markers comprise, but are not limited to, luciferase (e.g., bacterial, firefly and click beetle), luciferin, acquorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519, all of which are herein incorporated by reference in their entireties. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), all of which are herein incorporated by reference in their entireties. Labeling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, 6,649,138 and U.S. Pat. No. 6,815,064, all of which are herein incorporated by reference in their entireties. In some embodiments, a fluorescent label comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods are known for custom synthesis of nucleotides having other fluorophores (See, Henegariu et al. (2000) Nature Biotechnol. 18:345).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a oligonucleotide sequence is indirectly labeled, especially with a hapten that is then bound by a capture agent, e.g., as disclosed in U.S. Pat. Nos. 5,344,757, 5,702,888, 5,354,657, 5,198,537 and 4,849,336, and 5,073,562, all of which are herein incorporated by reference in their entireties. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some embodiments, the detectable label is a first detectable label, and the detectably labeled probe further comprises a second detectable label. In some embodiments, the first detectable label and the second detectable label are different. In some embodiments, detecting a complex comprising the detectably labeled probe in the biological sample comprises detecting a first signal from the first detectable label and detecting a second signal from the second detectable label. In some embodiments, the first detectable label is at the 5′ end of the detectably labeled probe and the second detectable label is at the 3′ end of the detectably labeled probe.

In some embodiments, the detectably labeled probe comprises a flexible linker between the reporter region and the first detectable label. In some embodiments, the flexible linker is a nucleotide sequence of between 1 and 10 nucleotides in length (e.g., between any of 2 and 10, 2 and 8, or 4 and 8 nucleotides in length). In some embodiments, the flexible linker is a non-nucleic acid linker. In some embodiments, the detectable label is linked to the reporter region by a disulfide.

In some embodiments, the detectable label is a fluorophore having an excitation peak between 480 nm and 500 nm. In some embodiments, the detectable label is a fluorophore having an excitation peak between 520 nm and 540 nm. In some embodiments, the detectable label is a fluorophore having an excitation peak between 590 nm and 600 nm. In some embodiments, the detectable label is a fluorophore having an excitation peak between 640 nm and 660 nm.

In some embodiments, the detected complex comprises at least 2 detectably labeled probes crosslinked to the second nucleic acid strand. In some embodiments, the detected complex comprises at least 2, 3, 4, 5, 6, or more detectably labeled probes crosslinked to the second nucleic acid strand. In some embodiments, the detected complex comprises between 2 and 20 detectably labeled probes crosslinked to the second nucleic acid strand. In some embodiments, the detected complex comprises at least 2, 3, 4, 5, 6, or more detectably labeled probes crosslinked to the second nucleic acid strand. In some embodiments, the detected complex comprises at least 2, 3, 4, 5, 6, or more detectably labeled probes crosslinked to the second nucleic acid strand. In some embodiments, the detected complex comprises at least 10, at least 20, at least 30, or at least 40 detectably labeled probe bound in the bNA structure. In some embodiments, the detected signal is the signal of at least 10, at least 20, at least 30, or at least 40 detectably labeled probes bound in the bNA structure.

### B. Crosslinkable Moieties and Intercalating Agents

In some embodiments, an oligonucleotide within the bNA structure provided herein comprises a crosslinkable moiety for interstrand crosslinking between the first nucleic acid, second nucleic acids, and detectably labeled probes. In some embodiments, the crosslinkable moiety is or is in a photoreactive nucleotide residue. In some embodiments, the crosslinkable moiety is a modified nucleotide amenable to click reaction chemistry. In some embodiments, the crosslinkable moiety is a modified nucleoside in the first nucleic acid strand, second nucleic acid strands, and/or detectably labeled probes. The linkage points in the bNA structure may comprise one or more crosslinkable moieties (e.g., photoreactive nucleotide residues). In some embodiments, crosslinking is performed to form interstrand crosslink between the first nucleic acid, and second nucleic acids. In some embodiments, crosslinking is performed to form interstrand crosslink between the second nucleic acids and the detectably labeled probes. In some embodiments, crosslinking is performed to form an interstrand crosslink between a plurality of the oligonucleotides of the first nucleic acid and second nucleic acids. In some embodiments, crosslinking is performed to form an interstrand crosslink between a plurality of the oligonucleotides of the second nucleic acids and detectably labeled probes. In some embodiments, the crosslinking occurs in the branch hybridization region of the first nucleic acid and second nucleic acids. In some embodiments, the crosslinking occurs in the reporter hybridization region of the second nucleic acids and detectably labeled probes. In some embodiments, the oligonucleotide of the second nucleic acid is crosslinked to the first nucleic acid upon activation by providing a stimulus. In some embodiments, the oligonucleotide of the second nucleic acid is crosslinked to the detectably labeled probe upon activation by providing a stimulus. In some embodiments, the oligonucleotide in the first nucleic acid strand is crosslinked to the second nucleic acid strand via the one or more crosslinkable moieties in the branch hybridization region. In some embodiments, the oligonucleotide in the detectably labeled probe is crosslinked to the second nucleic acid strand via the one or more crosslinkable moieties in the reporter region. The crosslinkable moiety or moieties may become photo-activated as described in below, in order to crosslink the oligonucleotide(s) within the assembled bNA structure.

In some embodiments, crosslinking is achieved by contacting the bNA structure with a crosslinkable intercalating agent such as a psoralen. Psoralens are molecules that can intercalate with nucleic acid (e.g., DNA) and that upon irradiation can form covalent bonds with pyrimidines (C/T/U). In the absence of irradiation, psoralens bind non-covalently similarly to any other intercalating agent, and covalent crosslinking to nucleic acid depends on irradiation. In some embodiments, the crosslinkable moiety comprises 8-methoxypsoralen (8-MOP), 5-methoxypsoralen (5-MOP), and/or 4,5′,8-trimethylpsoralen (TMP).

In some embodiments, the crosslinkable moiety is photocrosslinkable. In some embodiments, the crosslinkable moiety is an intercalating photoactive moiety within a nucleic acid duplex. In some embodiments, the crosslinkable moiety is a chemically modifying nucleic acid which is light-responsive. In some cases, irradiation with light can trigger photochemical reactions that can result in chemical ligation, reversal of chemical ligation, or nucleic acid dehybridization. In some embodiments, the crosslinkable moiety comprises a cyclobutane pyrimidine modification. In some embodiments, the crosslinkable moiety comprises a vinyl modification, such as cyanovinyl carbazole or derivative thereof. In some embodiments, the crosslinkable moiety comprises a carbazole modification. In some embodiments, the crosslinkable moiety comprises 5-carboxyvinyl-2′-deoxyuridine. In some embodiments, the crosslinkable moiety comprises a p-carbamoylvinyl phenol nucleoside. In some embodiments, the crosslinkable moiety comprises a cinnamate. In some embodiments, the crosslinkable moiety comprises an azobenzene or derivative thereof. Additional crosslinkable moieties can include but are not limited to those described in De Fazio et al., “Chemically modified nucleic acids and DNA intercalators as tools for nanoparticle assembly,” Chem. Soc. Rev., 2021, 50, 13410, which is incorporated herein by reference its entirety for all purposes.

In some embodiments, crosslinking is achieved through click reaction chemistry. Any suitable click reaction and click reactive groups may be used. In some embodiments, the click chemistry reaction consists of the condensation of organic azides with alkyne groups to form 1,2,3-triazole linkages. In some embodiments, crosslinking is achieved through copper catalyzed azide alkyne cycloaddition (CuAAC). In some embodiments, crosslinking is achieved through metal-free click chemistry. In some embodiments, crosslinking is achieved through strain promoted azide alkyne cycloaddition (SPAAC). In some embodiments, The SPAAC reaction involves the cycloaddition between a strained cyclooctyne and an organic azide.

In some embodiments, the linkage points comprise triazole linkages. In some embodiments, the linkage points comprise thiol linkages. In some embodiments, the linkage points are formed by amine-reactive chemistry.

In some embodiments, each of the linkage points is separated by at least 10 nucleotide residues. In some embodiments, the linkage point is separated by 10, 11, 12, 13, 14, or 15 nucleotides. In some embodiments, each of the linkage points is separated by between about 10 and about 20 nucleotide residues. In some embodiments, each of the linkage points are separated by between about 12 and about 20, about 14 and about 20, about 16 and about 20 nucleotide residues.

In some embodiments, the second nucleic acid strand comprises an overhang region functionalized with a plurality of acceptor moieties, wherein the method comprises covalently attaching the detectable moieties to the acceptor moieties to form the plurality of linkage points.

In some embodiments, the acceptor moieties comprise dibenzocyclooctyne (DBCO)- or alkyne-modified bases and the detectable moieties comprise an azide moiety; wherein the acceptor moieties comprise azide-modified bases and the detectable moieties comprise a DBCO or alkyne moiety. In some embodiments, the branch points on the first nucleic acid strand and second nucleic acid strand comprise an azide-modified base. In some embodiments, the acceptor moiety on the second nucleic acid strand and detectably labeled probe comprise an dibenzocyclooctyne (DBCO)- or alkyne-modified base. In some embodiments, covalently attaching the DBCO or alkyne modified bases and azide moieties comprises performing a copper-free or copper-catalyzed click chemistry reaction. In some embodiments, the covalently attaching the detectable moieties to the second nucleic acid strand comprises performing a copper-free or copper-catalyzed click chemistry reaction.

In some embodiments, activation of the crosslinkable moiety is light driven. In some embodiments, activation of the crosslinkable moiety is performed in aqueous solution. In some embodiments, crosslinking strands of nucleic acid molecules comprise at least one photo-reactive nucleobase. In some embodiments, the crosslinkable moiety is a photo-reactive nucleobase. In some embodiments, the photo-reactive nucleobase is any modified nucleobase that is capable of forming a crosslink with another nucleobase in an opposite hybridized strand in the presence of light. In some embodiments, the photo-reactive nucleobase is a modified pyrimidine or purine nucleobase. In some embodiments, the photo reactive nucleobase comprises a vinyl, acrylate, N-hydroxysuccinimide, amine, carboxylate or thiol chemical group. In some embodiments, the photo-reactive nucleobase comprises a bromo-deoxyuridine. Exemplary photoreactive crosslinkable moieties and photoreactive nucleotides are described, for example, in Elskens and Madder RSC Chem. Biol., 2021, 2, 410-422, the content of which is herein incorporated by reference in its entirety.

In some embodiments, the crosslinkable moiety comprises a reactive chemical group that requires light activation to initiate crosslinking. In some embodiments, the chemical group comprises, for example, an aryl azide, azido-methyl-coumarin, benzophenone, anthraquinone, certain diazo compounds, diazirine, or a psoralen derivative. In some embodiments, the psoralen derivative is selected from the group consisting of 8-methoxypsoralen (8-MOP), 5-methoxypsoralen (5-MOP) and 4,5′,8-trimethylpsoralen (TMP).

In some embodiments, the crosslinkable moiety comprises a cyanovinylcarbazole moiety. In some embodiments, the crosslinkable moiety is a vinylcarbazone-based moiety. In some embodiments, the crosslinkable moiety comprises a 3-cyanovinylcarbazole (^{CNV}K) nucleoside or 3-cyanovinylcarbazole modified D-threoninol (^{CMV}D). In some embodiments, the crosslinkable moiety comprises 3-cyanovinylcarbazole phosphoramidite. In some embodiments, the crosslinkable moiety comprises a pyranocarbazole. In some embodiments, the crosslinkable moiety comprises a pyranocarbazole (^{PC}X) modified nucleoside or a pyranocarbazole with a D-threoninol instead of a 2′-deoxyribose backbone (^{PCX}D). In some embodiments, the crosslinkable moiety comprises 5-carboxyvinyl-2′-deoxyuridine. In some embodiments, the crosslinkable moiety comprises a psoralen or a coumarin. In some embodiments, the photoreactive nucleotides have been attached to the oligonucleotide via a linker (e.g., a disulfide linker). In some embodiments, the crosslinkable moiety is a photoreactive nucleotide comprising a universal base. In some embodiments, the crosslinkable moiety is 5-bromo-2′-deoxyuridine (BrdU) or 5-bromo-2′-deoxycytidine (BrdC).

In some embodiments, the crosslinkable moiety is a pyranocarbazole (^{PC}X) modified nucleoside. The ^{PC}X crosslinking base displays high crosslinking efficiency with a thymine (T) base or a cytosine (C) base that is positioned adjacent to the base on the complementary strand and can be directly incorporated into the DNA hybridization domain itself as a base substitution. In some embodiments, a crosslinking reaction is performed using 400 nm wavelength of light and is completed within about 10 seconds. In some embodiments, a crosslinking reaction is completed within 0.1, 0.25, 0.5, 1, 5, or 10 seconds. In some embodiments, a crosslinking reaction is completed within 0.5, 1, 5, 10, 20, 30, 40, 50, or 60 minutes. In some embodiments, a crosslinking reaction is completed within 0.5, 1, 2, 3, 4, or 5 minutes. In some embodiments, a crosslinking reaction has negligible effects on bases that neighbor the photoreactive nucleobase. In some embodiments, other photochemical nucleic acid crosslinking agents, including psoralen can be used in combination with the photoreactive nucleobases disclosed herein. In some embodiments, a photo-induced crosslink is reversible. In some embodiments, a ^{PC}X crosslink is reversed when exposed to 312 or 305 nm UV light.

In some embodiments, other photochemical nucleic acid crosslinking agents, including psoralen and psoralen derivatives (e.g., psoralen modified nucleosides) are used as crosslinkable moieties. Psoralen and psoralen derivatives can be light-activated with a UV-A of 365 nm. Psoralens react with nearby pyrimidine residues. A variety of nucleosides modified with psoralen or psoralen derivatives may be used. For example, click chemistry using a psoralen azide and a nucleosidic alkyne derivative can be used to generate a variety of photoreactive nucleotides. In some embodiments, the psoralen is connected to the nucleotide via a linker, such as a phosphoramidite. Exemplary psoralen derivatives comprising phosphoramidite include but are not limited to 6-[4′-(Hydroxymethyl)-4,5′,8-trimethylpsoralen]-hexyl-1-O-(2-cyanoethyl)-(N,N-diisopropyl)-phosphoramidite and 2-[4′-(hydroxymethyl)-4,5′,8-trimethylpsoralen]-ethyl-1-O-(2-cyanoethyl)-(N,N-diisopropyl)-phosphoramidite. In some embodiments, the psoralen or psoralen derivative is conjugated to position 5 of a uridine or pseudouridine (optionally via a linker). In some cases, the psoralen or psoralen derivative is conjugated to the 2′ position of a sugar ring of a uridine or pseudouridine (optionally via a linker). In some embodiments, the psoralen derivative is an amine-reactive derivative. In some embodiments, an amine-reactive psoralen derivative is conjugated to an amine-modified nucleotide (e.g., an aminoallyl uridine or pseudouridine nucleotide).

In some embodiments, a psoralen-crosslink (e.g., an interstrand crosslink between the oligonucleotide and the hybridized intermediate probe) is reversed when exposed to 254 nm light. In some embodiments, the crosslinkable moiety comprises a C2′ psoralen modification. In some embodiments, the crosslinkable moiety comprises a 5′ psoralen derivative. In some embodiments, the crosslinkable moiety is at the 5′ end of the oligonucleotide. The structure of two exemplary psoralen-modified oligonucleotides (one 5′ modified nucleoside on the left, and one C2′ modified nucleoside on the right) are shown below:

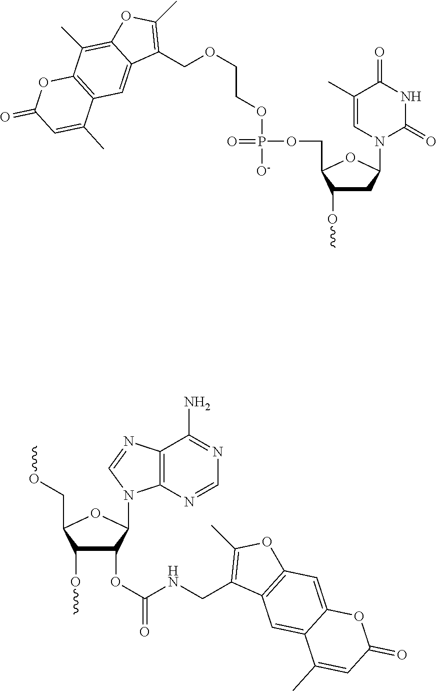

In some embodiments, the crosslinkable moiety is or is linked to a photoactivatable nucleotide, wherein the photoactivatable nucleotide is a universal base such as a pseudouridine modified with a photoreactive moiety (e.g. a psoralen).

In some embodiments, when the oligonucleotide comprises ^{CNV}K, rapid photo cross-linking to pyrimidines in the complementary strand (DNA or RNA) can be induced at one wavelength and rapid reversal of the cross-link is possible at a second wavelength if desired. Neither wavelength has the potential to cause significant DNA damage and neither interfere with the wavelengths used to excite the fluorophores used during subsequent analysis, such as decoding barcode sequences in situ. Once cross-linked, the UV melting temperature of the duplex may be raised by around 30° C./^{CNV}K moiety relative to the duplex before irradiation and inter-strand crosslinking. The structure of an exemplary 3-cyanovinylcarbazole phosphoramidite is shown below:

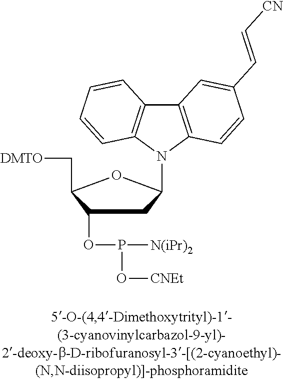

The ^{CNV}K crosslinking base displays high crosslinking efficiency with a thymine (T) base that is positioned adjacent to the base on the opposite hybridized strand in the target nucleic acid (e.g., the complementary strand) (Ultrafast reversible photo-cross-linking reaction: toward in situ DNA manipulation. Org. Lett. 10, 3227-3230 (2008)) and can be directly incorporated into the DNA hybridization domain itself as a base substitution, as shown below in light-directed reaction between a ^{CNV}K base modification and a thymine base to produce a crosslinked nucleic acid.

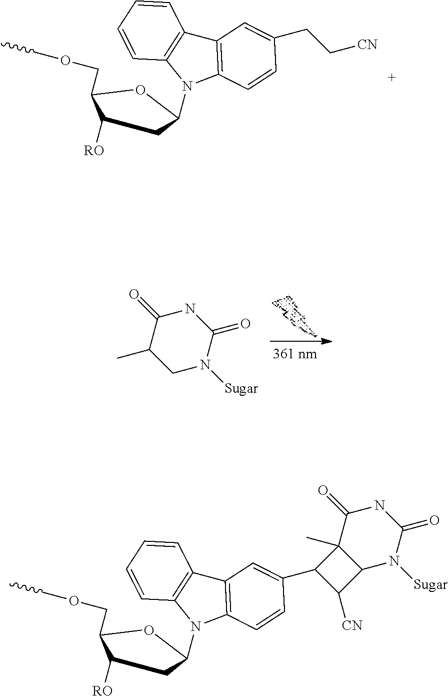

In some embodiments, a crosslinking reaction is performed using 365 nm wavelength of light. In some embodiments, the crosslinking reaction is completed within about 1 second. In some embodiments, a crosslinking reaction is performed using any suitable wavelength of visible or ultraviolet light based on the crosslinkable moiety used. In some embodiments, a crosslinking reaction is completed within 0.1, 0.25, 0.5, 1, 5, or 10 seconds. In some embodiments, a crosslinking reaction is completed within 20, 30, 40, 50, or 60 seconds. In some embodiments, the method comprises irradiating the biological sample with UV light, such as a 350-400 nm wavelength of light, for between 10 seconds and 10 minutes, between 10 seconds and 5 minutes, between 10 seconds and 2 minutes, between 10 seconds and 1 minute, between 30 seconds and 1 minute, or between 30 seconds and 5 minutes. In some embodiments, a crosslinking reaction is completed within 0.5, 1, 5, 10, 20, 30, 40, 50, or 60 minutes. In some embodiments, a crosslinking reaction has negligible effects on bases that neighbor the photoreactive nucleobase. In some embodiments, other photochemical nucleic acid crosslinking agents, including psoralen and coumarin are used in combination with the photoreactive nucleobases disclosed herein.

In some embodiments, the crosslinkable moiety comprises a coumarin and the photoactivation comprises irradiating the biological sample using a 350 nm wavelength of light. In some embodiments, the crosslinkable moiety comprises a psoralen and the photoactivation comprises irradiating the biological sample using a 365 nm wavelength of light. In some embodiments, the crosslinkable moiety comprises a ^{CNV}K or ^{CNV}D and the photoactivation comprises irradiating the biological sample using a 365 nm wavelength of light. In some embodiments, the crosslinkable moiety comprises a ^{PC}X or ^{PCX}D and the photoactivation comprises irradiating the biological sample using a 400 nm wavelength of light. In some embodiments, the crosslinkable moiety comprises a diazirine and the photoactivation comprises irradiating the biological sample using a 365 nm wavelength of light. In some embodiments, the crosslinkable moiety comprises a thiouridine and the photoactivation comprises irradiating the biological sample using a 365 nm wavelength of light.

In some embodiments, a photo-induced crosslink is reversible. In some embodiments, a vinylcarbazole (e.g., ^{CNV}K, ^{CNV}D, ^{PC}X, or ^{PCX}D) crosslink is reversed when exposed to 305 nm UV light. In some embodiments, a vinylcarbazole (e.g., ^{CNV}K, ^{CNV}D, ^{PC}X, or ^{PCX}D) crosslink is reversed when exposed to 312 nm light. In some embodiments, a psoralen crosslink is reversed when exposed to 254 nm light. In some embodiments, a coumarin crosslink is reversed when exposed to 254 nm light.

In some embodiments, the crosslinkable moiety is a photoactivatable nucleotide comprising a coumarin and hybridizes to a thymine (T) base in the complementary strand. In some embodiments, the photoactivatable nucleotide comprises a psoralen and hybridizes to a C, T, or U base in the complementary strand. In some embodiments, the photoactivatable nucleotide comprises a vinylcarbanazole and hybridizes to a C, T, or U base in the complementary strand. In some embodiments, the photoactivatable nucleotide comprises a universal or random base.

In some embodiments, the crosslinkable moiety crosslinks to an adenine (A) nucleobase in the strand of the target nucleic acid hybridized to the oligonucleotide. In some embodiments, the crosslinkable moiety comprises a psoralen capable of crosslinking to an adenine in the hybridized nucleic acid strand. In some embodiments, the crosslinkable moiety comprises a 5′-Dimethoxytrityl-2′-deoxy-4-(2-cyanoethylthio)-Thymidine,3′-[(2-cyanoethyl)-(N,N-diisopropyl)]-phosphoramidite (4-Thio-dT-CE phosphoramidite). In some embodiments, crosslinkable moiety comprises a 5′-Dimethoxytrityl-5-iodo-2′-deoxyUridine,3′-[(2-cyanoethyl)-(N,N-diisopropyl)]-phosphoramidite (5-I-dU-CE phosphoramidite).

The structure of an exemplary psoralen C2 phosphoramadite crosslinkable moiety is shown below:

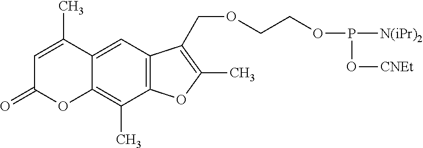

The structure of an exemplary 5′-Dimethoxytrityl-2′-deoxy-4-(2-cyanoethylthio)-Thymidine,3′-[(2-cyanoethyl)-(N,N-diisopropyl)]-phosphoramidite (4-Thio-dT-CE phosphoramidite) crosslinkable moiety is shown below:

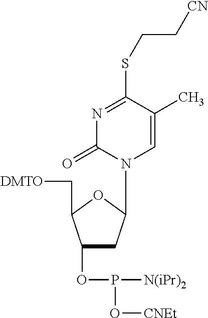

The structure of an exemplary 5′-Dimethoxytrityl-5-iodo-2′-deoxy Uridine,3′-[(2-cyanoethyl)-(N,N-diisopropyl)]-phosphoramidite (5-I-dU-CE phosphoramidite) crosslinkable moiety is shown below:

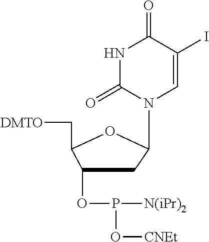

The photoreactive nucleotides may be photo-activated by UV light, such as a 350-400 nm wavelength of light, to photo-activate and crosslink the crosslinkable moiety of the hybridized second nucleic acid strands to the first nucleic acid strand and detectably labeled probes. In some embodiments, the crosslinkable moiety is crosslinked to the complementary strand at a 355 nm wavelength of light. In some embodiments, the purine bases of the target nucleic acid are unreactive to photo-activated crosslinking. In some embodiments, the pyrimidine bases of the complementary strand are reactive to photo-activated crosslinking. In some embodiments, the purine bases of the target nucleic acid are reactive to crosslinking (e.g., to a psoralen, 5-I-dU-CE, 4-Thio-dT-CE, or any other crosslinkable moiety configured to crosslink with nucleobases including adenine).

The photo-activated crosslinking step may be optimized to prevent DNA damage. In some embodiments, the photo-activated crosslinking does not cause significant DNA damage. In some embodiments, the photo-activated crosslinking between the hybridized second nucleic acid strands and the first nucleic acid strand, and/or between the second nucleic acid strands and the detectably labeled probes increases the UV melting temperature of the duplex compared to prior to the crosslinking. In some embodiments, the UV melting temperature is increased by about 30° C. per photoreactive nucleotide in the hybridization region.

### C. Crosslinking the bNA Structure

In some aspects the provided methods involve assembling the bNA structure and generating covalent linkages between the first nucleic acid strand, second nucleic acid strands, and detectably labeled probes. In some embodiments, prior to the contacting the biological sample, the method comprises generating the crosslinked bNA structure by providing a mixture comprising the first nucleic acid strand and the plurality of second nucleic acid strands and allowing the branch hybridization regions (BHR) to hybridize to the complementary branch hybridization region (BHR′), and irradiating the mixture to generate interstrand crosslinks between the branch hybridization regions (BHR) and the complementary branch hybridization region (BHR′). In some embodiments, generating the crosslinked bNA structure further comprises contacting the second nucleic acid strand with the plurality of detectably labeled probes and allowing the detectably labeled probes to hybridize to the reporter regions, and irradiating the mixture to generate interstrand crosslinks between the reporter regions and the hybridized detectably labeled probes. In some aspects, the provided methods allow for temporal control by controlling formation of the interstrand crosslinks.

In some embodiments, each of the DNA interstrand linkages is generated by UV-irradiation of a crosslinkable moiety. In some embodiments, the interstrand crosslink between the branch hybridization region (BHR) and the complementary branch hybridization region (BHR′) is generated by irradiating a crosslinkable moiety comprised by the branch hybridization region (BHR) and/or the complementary branch hybridization region (BHR′). In some embodiments, the interstrand crosslink between the reporter region and the complementary reporter hybridization region was generated by irradiating a crosslinkable moiety comprised by the reporter region and/or the complementary reporter hybridization region.

In some embodiments, the method comprises assembling the bNA structure by combining the first nucleic acid strand, second nucleic acid strands, and detectably labeled probes into one mixture. In some embodiments, the method comprises irradiating the mixture to photo-activate the crosslinkable moieties. In some embodiments, the first nucleic acid strand, second nucleic acid strands, and/or detectably labeled probes may or may not hybridize to form a complex (e.g., via nucleic acid hybridization) prior to photo-activation of the crosslinkable moieties. In some embodiments, the mixture is irradiated using a 350-400 nm wavelength of light. In some embodiments, the mixture is irradiated using a 366 nm wavelength of light. In some embodiments, the mixture is irradiated using a wavelength of light between about 350 nm to about 400 nm, about 360 nm to about 400 nm, about 370 nm to about 400 nm, about 380 nm to about 400 nm. In some embodiments, the mixture is irradiated using a 100-280 nm wavelength of light. In some embodiments, the mixture is irradiated using a wavelength of light between about 140 nm to about 280 nm, about 180 nm to about 280 nm, about 220 nm to about 280 nm, about 250 nm to about 280 nm.

In some embodiments, the method comprises assembling the bNA structure by combining one or more first nucleic acid strands, one or more second nucleic acid strands, and one or more detectably labeled probes into one mixture. In some embodiments, the method comprises assembling the bNA structure by combining a first nucleic acid strand, a plurality of second nucleic acid strands, and a plurality of detectably labeled probes into one mixture, wherein the plurality of second nucleic acid strands are each configured to be crosslinked to the first nucleic acid strand and the plurality of detectably labeled probes are each configured to be crosslinked to one of the second nucleic acid strands. In some embodiments, the method comprises irradiating the mixture to photoactivate the crosslinkable moieties for a specified amount of time. In some embodiments, the mixture is irradiated for between about 1 second to about 30 seconds. In some embodiments the mixture is irradiated for about 1 second, about 5 seconds, about 10 seconds, about 15 seconds, about 20 seconds, about 25 seconds, about 30 seconds, about 40 seconds, about 50 seconds or about 60 seconds. In some embodiments, the mixture is irradiated for 30 seconds. In some embodiments, the mixture is irradiated for no more than about 30 seconds, about 1 minute, about 2 minutes, about 3 minutes, about 4 minutes, or about 5 minutes. In some embodiments, the mixture is irradiated for no more than about 5 minutes. In some embodiments, the result of the photoactivation is to form a crosslinked bNA structure comprising one or more first nucleic acid strands, one or more second nucleic acid strands, and/or one or more detectably labeled probes that are covalently linked to one another, and in some aspects the interstrand linkages is reversible.

### D. Reversing Interstrand Crosslinks

In some aspects the provided methods involve decrosslinking the bNA structure. In some embodiments, after detecting the hybridized bNA structure, the method comprises irradiating the biological sample to reverse one or more of the interstrand linkages. In some embodiments, reversing one or more of the interstrand linkages destabilizes the bNA structure. In some embodiments, reversing one or more of the interstrand linkages allows the bNA structure to be cleared from the biological sample. In some embodiments, reversing one or more of the interstrand linkages allows the bNA structure to be cleared from the biological sample after a washing step. In some embodiments, the interstrand linkages are reversed and the bNA structure is removed from the biological sample.

In some embodiments, irradiating the biological sample to reverse one or more of the interstrand linkages comprises irradiating the sample with a wavelength between about 300 nm and about 320 nm. In some embodiments, reversing one or more of the interstrand linkages comprises irradiating the sample with a wavelength between about 305 nm and about 312 nm. In some embodiments, reversing one or more of the interstrand linkages comprises irradiating the sample with a wavelength between about 308 nm and about 320 nm, about 310 nm and about 320 nm, about 312 nm and about 320 nm, about 314 nm and about 320 nm, about 316 nm and about 320 nm, and about 318 nm and about 320 nm. In some embodiments, reversing one or more of the ^{CNV}K interstrand linkages comprises irradiating the sample with a wavelength between about 305 nm and about 312 nm.

In some embodiments, irradiating the biological sample to reverse one or more of the interstrand linkages comprises irradiating the sample with a wavelength between about 250 nm and about 300 nm. In some embodiments, reversing one or more of the interstrand linkages comprises irradiating the sample with a wavelength of between about 250 nm and about 290 nm, about 250 nm and about 280 nm, about 250 and about 270 nm, and about 250 and about 260 nm. In some embodiments, reversing one or more of the interstrand linkages comprises irradiating the sample with a wavelength of about 254 nm. In some embodiments, irradiating the biological sample to reverse one or more of the psoralen interstrand linkages comprises irradiating the sample with a wavelength between about 250 nm and about 300 nm.

In some embodiments, irradiating the biological sample to reverse one or more of the interstrand linkages comprises irradiating the sample for a specified amount of time. In some embodiments, reversing the one or more interstrand linkages comprises irradiating the biological sample for about 1 second to about 5 minutes. In some embodiments, reversing the one or more interstrand linkages comprises irradiating the biological sample for about 1 second, about 30 seconds, about 1 minute, about 2 minutes, about 3 minutes, about 4 minutes, or about 5 minutes. In some embodiments, reversing the one or more interstrand linkages comprises irradiating the biological sample for about 3 minutes. In some embodiments, reversing the one or more interstrand linkages comprises irradiating the biological sample for no more than about 1 minute, about 2 minutes, about 3 minutes, about 4 minutes, or about 5 minutes. In some embodiments, the mixture is irradiated for no more than about 5 minutes. In some embodiments, irradiating the biological sample leads to partial or complete reversal of the interstrand linkages in the crosslinked bNA structure comprising one or more first nucleic acid strands, one or more second nucleic acid strands, and/or one or more detectably labeled probes that are covalently linked to one another.

### E. Detection and Analysis

In some aspects, the provided methods involve analyzing, e.g., detecting or determining, one or more sequences present in a target nucleic acid molecule in the biological sample. In some embodiments, analyzing (e.g., detecting or determining) the one or more sequences in the target nucleic acid molecules comprises detecting the bNA structure comprising the detectably labeled probes. In some embodiments, the detecting is performed at one or more locations in the biological sample. In some embodiments, the locations are the locations of RNA transcripts in the biological sample. In some embodiments, the locations are the locations at which the probes or probe sets hybridize to the RNA transcripts in the biological sample, and are optionally ligated and amplified by rolling circle amplification. In some embodiments, the probes or probe sets hybridized to the RNA transcripts in the biological sample are not amplified by rolling circle amplification.

In some embodiments, the detecting comprises a plurality of sequential cycles of bNA complex hybridization and removal. For example, in some embodiments the detecting comprises a plurality of sequential cycles of binding and removal of bNA structures (e.g., crosslinked first nucleic acid strand, second nucleic acid strand, and detectably labeled probes) to the primary probe or probe set hybridized to the target nucleic acid, optionally wherein the bNA structures bind to the primary probe indirectly (via a probe or probe set). In some embodiments, the detecting comprises a plurality of sequential cycles of binding and removal of bNA structures (e.g., crosslinked first nucleic acid strand, second nucleic acid strand, and detectably labeled probes) to the target nucleic acid, optionally wherein bNA structures bind to the primary probe indirectly (via a probe or probe set). In some embodiments, the target nucleic acid is a nucleic acid analyte. In some embodiments, the target nucleic acid is a probe or a product provided as a proxy for a nucleic acid analyte (e.g., a rolling circle amplification product generated from a circular or circularized probe that binds directly or indirectly to the nucleic acid analyte). In some embodiments, the target nucleic acid is a nucleic acid molecule provided as a proxy for a non-nucleic acid analyte (e.g., an oligonucleotide reporter in a labeling agent that binds to the non-nucleic acid analyte, or a product such as a rolling circle amplification product associated with the oligonucleotide reporter).

In some embodiments, the detecting comprises a plurality of sequential cycles of bNA complex hybridization removal of at least a portion of the bNA complex comprising the detectable moieties (e.g., by cleaving the bNA complex to remove the detectable moieties and/or reversing the crosslinking of a reversible crosslinkable moiety and washing away the detectable probes). In some embodiments, cleaving the bNA complex comprises an enzymatic or chemical cleavage. In some embodiments, cleaving the bNA complex comprises cleaving one or more thiol linkages in the bNA complex. In some embodiments, cleaving the bNA complex comprises the use of reducing agents such as Dithiothreitol (DTT) or TCEP (tris(2-carboxyethyl) phosphine). In some instances, a portion of the bNA complex is removed by cleaving (e.g., using an enzymatic or chemical cleavage) at a position in the first nucleic acid strand.

In some embodiments, a plurality of species of crosslinked bNA structures comprising different detectable moieties (e.g., different color fluorophores) are separately pre-assembled and then pooled to form a bNA complex mixture. In some embodiments, the bNA complex mixture comprises 2, 3, 4, or more different species of bNA (e.g., comprising different color fluorophores). In some embodiments, the methods provided herein comprise contacting the biological sample with the pooled mixture of bNA complexes.

Methods for binding and identifying a target nucleic acid that uses various probes or oligonucleotides have been described in, e.g., U.S. Pat. Nos. 7,473,767, 7,919,237, US2010/0015607, U.S. Pat. Nos. 8,986,926, 7,941,279, 8,415,102, 8,519,115, and US2014/0371088, each of which is incorporated herein by reference in its entirety. In some embodiments, detectably-labeled probes are useful for detecting multiple target nucleic acids and are detected in one or more hybridization cycles (e.g., sequential hybridization assays, or sequencing by hybridization).

In some embodiments, the detecting comprises binding a bNA structure directly or indirectly (e.g., via an adapter) to the primary probe or probe set, and detecting a signal associated with the detectably labeled probe. In some embodiments, the method can comprise performing one or more wash steps to remove unbound and/or nonspecifically bound bNA structures.

In some embodiments, the method comprises detecting a reporter region in a bNA structure by sequencing all or a portion of the reporter region. In some embodiments, the method comprises detecting a reporter region in a bNA structure by sequencing all or a portion of the reporter region at a location in the biological sample. In some embodiments, the method comprises contacting the bNA structure with a sequencing primer that binds adjacent to the reporter region. In some embodiments, sequence analysis is performed by using a base-by-base sequencing method, e.g., sequencing-by-synthesis (SBS), sequencing-by-avidity (SBA), or sequencing-by-binding (SBB). In some embodiments, the sequence to be analyzed is contacted with a sequencing primer and base-by-base sequencing using a cyclic series of nucleotide incorporation or binding, respectively, thereby generating extension products of the sequencing primer is performed followed by removing, cleaving, or blocking the extension products of the sequencing primer.

In some embodiments, for sequencing-by-synthesis methods, a first population of detectably labeled nucleotides (e.g., dNTPs) are introduced to contact a template nucleotide (e.g., a sequence in the bNA structure) hybridized to a sequencing primer, and a first detectably labeled nucleotide (e.g., A, T, C, or G nucleotide) is incorporated by a polymerase to extend the sequencing primer in the 5′ to 3′ direction using a complementary nucleotide (a first nucleotide residue) in the template nucleotide as template. A signal from the first detectably labeled nucleotide can then be detected. The first population of nucleotides may be continuously introduced, but in order for a second detectably labeled nucleotide to incorporate into the extended sequencing primer, nucleotides in the first population of nucleotides that have not incorporated into a sequencing primer are generally removed (e.g., by washing), and a second population of detectably labeled nucleotides are introduced into the reaction. Then, a second detectably labeled nucleotide (e.g., A, T, C, or G nucleotide) is incorporated by the same or a different polymerase to extend the already extended sequencing primer in the 5′ to 3′ direction using a complementary nucleotide (a second nucleotide residue) in the template nucleotide as template. Thus, in some embodiments, cycles of introducing and removing detectably labeled nucleotides are performed.

In some embodiments, the base-by-base sequencing comprises using a polymerase that is fluorescently labeled. In some embodiments, the base-by-base sequencing comprises using a polymerase-nucleotide conjugate comprising a fluorescently labeled polymerase linked to a nucleotide moiety that is not fluorescently labeled. In some embodiments, the base-by-base sequencing comprises using a multivalent polymer-nucleotide conjugate comprising a polymer core, multiple nucleotide moieties, and one or more fluorescent labels.

In some embodiments, sequencing is performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Example SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequencing is performed by sequencing-by-binding (SBB). Various aspects of SBB are described in U.S. Pat. No. 10,655,176 B2, the content of which is herein incorporated by reference in its entirety. In some embodiments, SBB comprises performing repetitive cycles of detecting a stabilized complex that forms at each position along the template nucleic acid to be sequenced (e.g. a ternary complex that includes the primed template nucleic acid, a polymerase, and a cognate nucleotide for the position), under conditions that prevent covalent incorporation of the cognate nucleotide into the primer, and then extending the primer to allow detection of the next position along the template nucleic acid. In the sequencing-by-binding approach, detection of the nucleotide at each position of the template occurs prior to extension of the primer to the next position. Generally, the methodology is used to distinguish the four different nucleotide types that can be present at positions along a nucleic acid template by uniquely labelling each type of ternary complex (i.e. different types of ternary complexes differing in the type of nucleotide it contains) or by separately delivering the reagents needed to form each type of ternary complex. In some instances, the labeling may comprise fluorescence labeling of, e.g., the cognate nucleotide or the polymerase that participate in the ternary complex.

In some embodiments, sequencing is performed by sequencing-by-avidity (SBA). Some aspects of SBA approaches are described in U.S. Pat. No. 10,768,173 B2, the content of which is herein incorporated by reference in its entirety. In some embodiments, SBA comprises detecting a multivalent binding complex formed between a fluorescently-labeled polymer-nucleotide conjugate, and a one or more primed target nucleic acid sequences (e.g., barcode sequences). Fluorescence imaging is used to detect the bound complex and thereby determine the identity of the N+1 nucleotide in the target nucleic acid sequence (where the primer extension strand is N nucleotides in length). Following the imaging step, the multivalent binding complex is disrupted and washed away, the correct blocked nucleotide is incorporated into the primer extension strand, and the sequencing cycle is repeated.

In some aspects, the detecting comprises imaging the biological sample. In some embodiments, the bNA structure is stable during the imaging. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity-so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECS™), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PS™, photon scanning tunneling microscopy (PS™), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXS™), and intact tissue expansion microscopy (exM).

In some embodiments, the bNA structure is bound to a target sequence in a target nucleic acid, wherein the target sequence is a barcode sequence. In some embodiments, sequential hybridization of bNA structures comprising first nucleic acid strands, second nucleic acid strands, and detectably labeled probes bound to the target sequence (e.g., barcode sequence) is used to detect the barcode sequence. In some embodiments, the method comprises forming a first complex comprising hybridizing a first bNA structure bound directly or indirectly to the target sequence and imaging the biological sample, removing the first bNA structure, and hybridizing a second bNA structure to a target nucleic acid in the biological sample. In some embodiments, the bNA structure is a first bNA structure, and detecting the first bNA structure comprises detecting a first signal of a signal code assigned to the target nucleic acid, wherein after detecting the hybridized first bNA structure at a location in the biological sample, the method comprises: (c) removing the first bNA structure, (d) contacting the biological sample with a second bNA structure, wherein the second bNA structure binds directly or indirectly to the target nucleic acid, and (e) detecting the second bNA structure, wherein detecting the second bNA structure comprises detecting a second signal of the signal code assigned to the target nucleic acid. In some embodiments, the first signal is a first signal code of a signal code sequence (a temporal “barcode” formed from a temporally sequential series of signals, such as a series of fluorescent colors) that can be used to identify the target nucleic acid molecule (e.g., by identifying the target sequence such as a nucleotide barcode sequence in the target nucleic acid molecule). In some embodiments, the signal code sequence identifies an analyte associated with the target nucleic acid molecule (e.g., a nucleic acid analyte or a non-nucleic acid analyte). The biological sample can be sequentially contacted with pools of pre-assembled bNA structures any number of times to detect sufficient signals to identify the target sequence.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds. In some embodiments, the analysis and/or sequence determination involves washing to remove unbound polynucleotides, thereafter revealing a fluorescent product for imaging.

In some embodiments, the method comprises imaging the biological sample at an excitation wavelength between 312 nm and 358 nm to detect a nuclear stain. In some embodiments, the detecting a nuclear stain comprises an excitation wavelength between about 310 nm and about 360 nm, about 340 nm and about 360 nm, about 360 nm and about 380 nm. In some embodiments the nuclear stain is comprised of 4′,6-diamidino-2-phenylindole (DAPI), or Hoechst dyes. In some embodiments the nuclear stain is DAPI.

In some embodiments, reversing the one or more of the interstrand linkages occurs during the imaging of the nuclear stain. In some embodiments, imaging the biological sample at an excitation wavelength between 312 nm and 358 nm is performed after detecting the bNA structure at the location in the biological sample. In some embodiments, the method comprises washing the biological sample to remove the bNA structure after detecting the bNA structure in the biological sample. In some embodiments, the method comprises reversing one or more of the interstrand linkages prior to washing the biological sample to remove the bNA structure after detecting the bNA structure in the biological sample.

### III. Biological Samples and Sample Preparation

### A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, biopsy, a cell block, a cell pellet, a core biopsy, needle aspirate, or fine needle aspirate. The sample can be a fluid sample, such as a blood sample, urine sample, or saliva sample. The sample can be a skin sample, a colon sample, a check swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface, or cells from a portion of a cell block or cell pellet.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some embodiments, a substrate herein is any support that is insoluble in aqueous liquid and which allows for positioning of biological samples, analytes, features, and/or reagents (e.g., probes) on the support. In some embodiments, a biological sample is attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Preparation

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than-25° C.

In some embodiments, the biological sample is prepared using formalin-fixation and paraffin-embedding (FFPE), which are established methods. In some embodiments, cell suspensions and other non-tissue samples are prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes). In some embodiments, the biological sample (e.g., FFPE sample) is permeable after deparaffinization.

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe and a target is formed in a sample. In some embodiments, one or more post-fixing step is performed prior to a ligation reaction disclosed herein.

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

In some embodiments, a biological sample is permeabilized to facilitate transfer of species (such as probes) into the sample. In some embodiments, processing of the biological sample, such as de-waxing, allows the biological sample to become permeabilized. If a sample is not permeabilized sufficiently, the transfer of species (such as probes and/or bNA structure as described herein) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample is incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample is permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe or nucleic acid strands of a bNA structure. For example, proteinase K treatment may be used to free up DNA with proteins bound thereto.

### (ii) Embedding

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample. Biological samples can include analytes (e.g., protein, RNA, and/or DNA) embedded in a 3D matrix. In some embodiments, amplification products (e.g., hybridized bNA structures or rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some aspects, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof are modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347 (6221): 543-548, 2015, the entire contents of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry is used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible. In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some embodiments, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a biological sample embedded in a matrix (e.g., a hydrogel) is isometrically expanded. Isometric expansion methods that can be used include hydration, a preparative step in expansion microscopy, as described in, e.g., Chen et al., Science 347 (6221): 543-548, 2015 and U.S. Pat. No. 10,059,990, which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(iii) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, cosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and cosin (H&E).

The sample can be stained using hematoxylin and cosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples are destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65 (8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### B. Analytes

A biological sample may comprise one or a plurality of analytes of interest. Methods for performing multiplexed assays to analyze two or more different analytes in a single biological sample using any of the bDNA structures disclosed herein are provided. The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### (i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.)

### (ii) Labeling Agents

In some embodiments, provided herein are methods and compositions for analyzing endogenous analytes (e.g., RNA, ssDNA, cell surface or intracellular proteins, and/or metabolites) in a sample using any of the bNA structures disclosed herein, and one or more labeling agents. In some embodiments, the bNA structure binds directly or indirectly to a labeling agent. In some embodiments, an analyte labeling agent may include an agent that interacts with an analyte (e.g., an endogenous analyte in a sample). In some embodiments, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the bNA structure binds directly or indirectly to the probe. In some embodiments, the analyte labeling agent comprises an analyte binding moiety and a labeling agent barcode domain comprising one or more barcode sequences, e.g., a barcode sequence that corresponds to the analyte binding moiety and/or the analyte. In some embodiments, the bNA structure binds to the labeling agent barcode domain (e.g., to a barcode sequence or subunit thereof in the labeling agent barcode domain). An analyte binding moiety barcode includes a barcode that is associated with or otherwise identifies the analyte binding moiety. In some embodiments, by identifying an analyte binding moiety by identifying its associated analyte binding moiety barcode, the analyte to which the analyte binding moiety binds can also be identified. Thus, a method of analyzing an analyte moiety barcode sequence by sequential hybridization of one or more bNA structures to the barcode sequence or subunits thereof can be used to identify the analyte. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labeling agents. In some embodiments, the post-fixing steps are performed before contacting the sample with the bNA structure. In some embodiments, the post-fixing steps are performed after contacting the sample with the bNA structure.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. No. 10,725,027, which are each incorporated by reference herein in their entirety.

In some embodiments, an analyte binding moiety includes one or more antibodies or epitope-binding fragments thereof. The antibodies or epitope-binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31 (2): 708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent comprises a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample are subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.
(iii) Generation of Products

In some embodiments, provided herein are methods and compositions for analyzing one or more products of an endogenous analyte and/or a labeling agent in a biological sample using any of the bNA structures disclosed herein. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labeling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product of a labeling agent that directly or indirectly binds to an analyte in the biological sample is analyzed.

### (a) Hybridization

In some embodiments, a hybridization product comprising the pairing of substantially complementary or complementary nucleic acid sequences within two different molecules can be analyzed using any of the bNA structures disclosed herein. For example, hybridization of an endogenous analyte or the labeling agent (e.g., reporter oligonucleotide attached thereto) with another endogenous molecule or another labeling agent or a probe can be analyzed. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labeling agent and each probe may comprise one or more barcode sequences. Exemplary barcoded probes or probe sets may be based on a circularizable probe e.g., a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

### (b) Ligation

In some embodiments, a ligation product of an endogenous analyte and/or a labeling agent can be analyzed using the bNA structures disclosed herein. In some embodiments, the ligation product is formed between two or more endogenous analytes. In some embodiments, the ligation product is formed between two or more labeling agents. In some embodiments, the ligation product is an intramolecular ligation of an endogenous analyte. In some embodiments, the ligation product is an intramolecular ligation product or an intermolecular ligation product, for example, the ligation product can be generated by the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labeling agent (e.g., the reporter oligonucleotide) or a product thereof.

In some embodiments, the ligation involves chemical ligation (e.g., click chemistry ligation). In some embodiments, the chemical ligation involves template dependent ligation. In some embodiments, the chemical ligation involves template independent ligation. In some embodiments, the click reaction is a template-independent reaction (see, e.g., Xiong and Seela (2011), J. Org. Chem. 76 (14): 5584-5597, incorporated by reference herein in its entirety). In some embodiments, the click reaction is a template-dependent reaction or template-directed reaction. In some embodiments, the template-dependent reaction is sensitive to base pair mismatches such that reaction rate is significantly higher for matched versus unmatched templates. In some embodiments, the click reaction is a nucleophilic addition template-dependent reaction. In some embodiments, the click reaction is a cyclopropane-tetrazine template-dependent reaction.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo) nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo) nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo) nucleotide, such that the gap (oligo) nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

In some embodiments, the bNA structures provided herein bind directly or indirectly to a probe or probe set capable of DNA-templated ligation, such as from a cDNA molecule. Sec, e.g., U.S. Pat. No. 8,551,710, which is hereby incorporated by reference in its entirety. In some embodiments, the bNA structures provided herein bind directly or indirectly to a probe or probe set capable of RNA-templated ligation. See, e.g., U.S. Pat. No. 11,597,965 which is hereby incorporated by reference in its entirety. In some embodiments, the bNA structures provided herein bind directly or indirectly to a product of a probe or probe set, such as a rolling circle amplification product formed from a circularized probe or probe set, optionally wherein the probe or probe set is circularized by one or more ligations. In some embodiments, the probe set is a SNAIL probe set. Sec, e.g., U.S. Pat. No. 11,008,608, which is hereby incorporated by reference in its entirety. In some embodiments, the bNA structures provided herein are used to detect a probe or probe set or product thereof in a multiplexed proximity ligation assay. Exemplary multiplexed proximity ligation assays are described in U.S. Pat. No. 10,465,235, which is hereby incorporated by reference in its entirety. In some embodiments, the bNA structures provided herein are used to detect a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, the bNA structures provided herein are used to detect a circular probe or product thereof (such as a rolling circle amplification product of a circular probe). In some embodiments, a circular probe can be indirectly hybridized to the target nucleic acid. In some embodiments, the circular construct is formed from a probe set capable of proximity ligation, for instance a proximity ligation in situ hybridization (PLISH) probe set. Sec, e.g., U.S. Pat. Pub. 2020/0224243 which is hereby incorporated by reference in its entirety.

### (c) Primer Extension and Amplification

In some embodiments, a primer extension product of an analyte, a labeling agent, a probe or probe set bound to the analyte (e.g., a circularizable probe bound to genomic DNA, mRNA, or cDNA), or a probe or probe set bound to the labeling agent (e.g., a circularizable probe bound to one or more reporter oligonucleotides from the same or different labeling agents) can be analyzed using any of the bNA structures and/or methods disclosed herein.

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labeling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) include linear RCA, a branched RCA, a dendritic RCA, or any combination thereof. (Sec, e.g., Baner et al, Nucleic Acids Research, 26:5073-5078, 1998; Lizardi et al, Nature Genetics 19:226, 1998; Mohsen et al., Acc Chem Res. 2016 Nov. 15; 49 (11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, WO 2014/163886, WO 2017/079406, U.S. Pat. Nos. 10,138,509, 10,266,888 and US 2017/0219465, which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products (e.g., hybridized bNA structures) are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step, functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. In some embodiments, different analytes are detected in situ in one or more cells using amplification (e.g., by hybridizing a bNA structure or a RCA-based detection system, e.g., where the signal is provided by generating an RCA product from a circular RCA template which is provided or generated in the assay, and the RCA product is detected to detect the corresponding analyte). The hybridized bNA structure or RCA product may thus be regarded as a reporter which is detected to detect the target analyte. However, the RCA template may also be regarded as a reporter for the target analyte; the RCA product is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a bNA structure disclosed herein may be a hybridization complex formed of a cellular nucleic acid in a sample and an exogenously added nucleic acid probe. The exogenously added nucleic acid probe may comprise an overhang that does not hybridize to the cellular nucleic acid but hybridizes to a first nucleic acid strand of the bNA structure. The exogenously added nucleic acid probe may be optionally ligated to a cellular nucleic acid molecule or another exogenous nucleic acid molecule. In other examples, a product comprising a target sequence for a bNA structure disclosed herein may be an RCP of a circularizable probe or probe set which hybridizes to a cellular nucleic acid molecule (e.g., genomic DNA or mRNA) or product thereof (e.g., a transcript such as cDNA, a DNA-templated ligation product of two probes, or an RNA-templated ligation product of two probes). In other examples, a product comprising a target sequence for a bNA structure disclosed herein may a probe hybridizing to an RCP. The probe may comprise an overhang that does not hybridize to the RCP but hybridizes to a first nucleic acid strand of the bNA structure. The probe may be optionally ligated to a cellular nucleic acid molecule or another probe, e.g., an anchor probe that hybridizes to the RCP.

### C. Barcode Sequences

In some embodiments, an analyte described herein is associated with one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides. In some embodiments, the adapter region of a probe in a bNA structure (e.g., as described in Section II) is or comprises a barcode sequence or subunit thereof.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide. In any of the preceding embodiments, the methods provided herein can include analyzing the barcodes by sequential hybridization and detection with a plurality of labelled probes (e.g., detection oligos).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4˜ complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (45=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. No. 11,008,608 and U.S. Pat. Pub 20210164039, which are hereby incorporated by reference in their entirety.

### VI. Compositions and Kits

In some aspects, provided herein are kits for analyzing an analyte in a biological sample according to any of the methods described herein. In some embodiments, the kit comprises assembled bNA structures. In some embodiments, provided herein is a kit comprising (i) a first nucleic acid strand comprising an adapter hybridization region that hybridizes to an adapter region in a target nucleic acid or in a probe or probe set hybridized to a target nucleic acid in the biological sample and a plurality of branch hybridization regions (BHRs), and (ii) a plurality of second nucleic acid strands comprising a complementary branch hybridization region (BHR′) and a detectable label or an overhang region comprising one or more reporter regions for directly or indirectly binding to one or more detectably labeled probes, wherein BHR′ hybridizes to BHR, wherein at least a subset of the second nucleic acid strands are covalently attached to the first nucleic acid strand via an interstrand crosslink in the BHR, (iii) a plurality of detectably labeled probes comprising a reporter hybridization region and a detectable label.

In some embodiments, the first nucleic acid strand comprises 2, 3, 4, 5, 6, or more BHRs. In some embodiments, the first nucleic acid comprises between 2 and 20 BHRs. In some embodiments, the first nucleic acid comprises between 2 and 4, between 2 and 5, or between 2 and 6 BHRs. In some embodiments, the first nucleic acid strand comprises 3 BHRs. In some embodiments, the first nucleic acid strand comprises 4 BHRs. In some embodiments, the at least two BHRs are separated by a spacer region of at least 4 nucleotides in length. In some embodiments, the spacer region is at least 5, at least 6, at least 7, or at least 8 nucleotides in length. In some embodiments, the spacer region is no more than 10, no more than 9, no more than 8, or no more than 7 nucleotides in length. In some embodiments, the spacer region is between 4 and 10, between 4 and 9, between 4 and 8, or between 5 and 8 nucleotides in length. In some embodiments, the spacer region is between 4 and 20, between 4 and 15, between 4 and 10, between 8 and 20, or between 8 and 15 nucleotides in length. In some embodiments, each of the spacer regions has the same sequence. In some embodiments, the spacer regions have different sequences. In some embodiments, the spacer regions have random sequences. In some embodiments, the spacer region sequences are a sequence of adenines and/or thymidines. In some embodiments, each of the BHRs has the same sequence (e.g., the BHRs are copies of the same sequence to allow hybridization of multiple molecules of the same second nucleic acid strand to the first nucleic acid strand).

In some embodiments, the first nucleic acid strand comprises, from 5′ to 3′ or from 3′ to 5′, an adapter hybridization region, a spacer region, and a first BHR, a spacer region, and a second BHR. In some embodiments, the first nucleic acid strand comprises, from 5′ to 3′ or from 3′ to 5′, an adapter hybridization region, a spacer region, and a first BHR, a spacer region, and a second BHR, a spacer region, a third BHR, a spacer region, and a fourth BHR. In some embodiments, the first, second, third, and fourth BHRs are individually between 15 and 25 nucleotides in length, between 16 and 25 nucleotides in length, between 16 and 22 nucleotides in length, or between 16 and 20 nucleotides in length. In some embodiments, the BHRs are individually about 16 nucleotides in length. FIG. 2A illustrates an exemplary first nucleic acid strand comprising an adapter hybridization region and three BHRs.

In some embodiments, the second nucleic acid strands comprises an overhang region comprising a plurality of reporter regions, wherein the bNA structure comprises a plurality of detectably labeled probes hybridized to the reporter regions via complementary reporter hybridization regions, optionally wherein at least a subset of the reporter regions are covalently attached to the hybridized detectably labeled probes via an interstrand crosslink.

In some embodiments, the second nucleic acid strand comprises 2, 3, 4, 5, 6, or more reporter regions. In some embodiments, the second nucleic acid comprises between 2 and 20 reporter regions. In some embodiments, the second nucleic acid comprises between 2 and 4, between 2 and 5, or between 2 and 6 reporter regions. In some embodiments, the second nucleic acid strand comprises 3 reporter regions. In some embodiments, the second nucleic acid strand comprises 4 reporter regions. In some embodiments, the at least two reporter regions are separated by a spacer region of at least 4 nucleotides in length. In some embodiments, the spacer region is at least 5, at least 6, at least 7, or at least 8 nucleotides in length. In some embodiments, the spacer region is no more than 10, no more than 9, no more than 8, or no more than 7 nucleotides in length. In some embodiments, the spacer region is between 4 and 10, between 4 and 9, between 4 and 8, or between 5 and 8 nucleotides in length. In some embodiments, the spacer region is between 4 and 20, between 4 and 15, between 4 and 10, between 8 and 20, or between 8 and 15 nucleotides in length. In some embodiments, each of the spacer regions has the same sequence. In some embodiments, the spacer regions have different sequences. In some embodiments, the spacer regions have random sequences. In some embodiments, the spacer region sequences are a sequence of adenines and/or thymidines. In some embodiments, each of the reporter regions has the same sequence (e.g., the reporter regions are copies of the same sequence to allow hybridization of multiple molecules of the same detectably labeled probes to the second nucleic acid strand).

In some embodiments, a detectably labeled probe herein comprises a reporter hybridization region and a detectable label. In some embodiments, the detectably labeled probes are individually between 15 and 25 nucleotides in length, between 15 and 30 nucleotides in length, between 15 and 40 nucleotides in length, or between 20 and 40 nucleotides in length. In some embodiments the reporter hybridization region contains complementarity with the reporter region in the second nucleic acid strands. In some embodiments, the reporter hybridization region is between 15 and 25 nucleotides in length, between 15 and 20 nucleotides in length, between 16 and 25 nucleotides in length, between 16 and 22 nucleotides in length, or between 20 and 25 nucleotides in length. In some embodiments, the reporter hybridization region is no more than any one of 16, 17, 18, 19, 20, 22, or 25 nucleotides in length.

In some embodiments, each of the second nucleic acid strands are covalently linked to a plurality of detectable moieties at a plurality of linkage points. In some embodiments, linkage points consist of a crosslinkable moiety within the reporter regions of the second nucleic acid strands, or branch points within the second nucleic acid strands.

In some embodiments, the detectably labeled probe herein comprises a branch point acceptor and a detectable label. In some embodiments, the branch point acceptor is a modified crosslinkable nucleotide comprising a moiety that can exclusively react with a branch point modified nucleotide within the second nucleic acid strand. In some embodiments, the branch point acceptor is a modified crosslinkable nucleotide comprising a moiety that can exclusively react with a branch point modified nucleotide within the second nucleic acid strand, wherein the reaction comprises click reaction chemistry. In some embodiments the reaction between the branch point acceptor and the branch point results in a covalent crosslink between the second nucleic acid strand and the detectably labeled probe. In some embodiments the detectably labeled probes comprising a branch point acceptor and a detectable label are individually between 5 and 25 nucleotides in length, between 5 and 30 nucleotides in length, between 5 and 40 nucleotides in length, or between 2 and 40 nucleotides in length. In some embodiments, the reporter region is between 10 and 25 nucleotides in length, between 10 and 20 nucleotides in length, between 12 and 25 nucleotides in length, between 16 and 22 nucleotides in length, or between 20 and 25 nucleotides in length. In some embodiments, the reporter region is no more than any one of 12, 14, 16, 18, 20, 22, or 25 nucleotides in length.

In some embodiments, the detectable label is a label that can be measured and quantitated. The detectable label can comprise a directly or indirectly detectable moiety, e.g., any fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

In some embodiments, the detectable label is a fluorophore. A fluorophore can comprise a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. Background fluorescence can include autofluorescence (that can arise from a variety of sources, including aldehyde fixation, extracellular matrix components, red blood cells, lipofuscin, and the like), as opposed to the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (Max Vision Biosciences), and/or a very intense black dye (e.g., Sudan Black, or comparable dark chromophore).

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, acquorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519, all of which are herein incorporated by reference in their entireties. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), all of which are herein incorporated by reference in their entireties. Labeling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, 6,649,138 and 6,815,064, all of which are herein incorporated by reference in their entireties. In some embodiments, a fluorescent label comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods are known for custom synthesis of nucleotides having other fluorophores (See, Henegariu et al. (2000) Nature Biotechnol. 18:345).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a oligonucleotide sequence is indirectly labeled, especially with a hapten that is then bound by a capture agent, e.g., as disclosed in U.S. Pat. Nos. 5,344,757, 5,702,888, 5,354,657, 5,198,537 and 4,849,336, and 5,073,562, all of which are herein incorporated by reference in their entireties. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some embodiments, the detectable label is a first detectable label, and the detectably labeled probe further comprises a second detectable label. In some embodiments, the first detectable label and the second detectable label are different. In some embodiments, detecting a complex comprising the detectably labeled probe in the biological sample comprises detecting a first signal from the first detectable label and detecting a second signal from the second detectable label. In some embodiments, the first detectable label is at the 5′ end of the detectably labeled probe and the second detectable label is at the 3′ end of the detectably labeled probe.

In some embodiments, the detectably labeled probe comprises a flexible linker between the reporter region and the first detectable label. In some embodiments, the flexible linker is a nucleotide sequence of between 1 and 10 nucleotides in length (e.g., between any of 2 and 10, 2 and 8, or 4 and 8 nucleotides in length). In some embodiments, the flexible linker is a non-nucleic acid linker. In some embodiments, the detectable label is linked to the reporter region by a disulfide.

In some embodiments, the detectable label is a fluorophore having an excitation peak between 480 nm and 500 nm. In some embodiments, the detectable label is a fluorophore having an excitation peak between 520 nm and 540 nm. In some embodiments, the detectable label is a fluorophore having an excitation peak between 590 nm and 600 nm. In some embodiments, the detectable label is a fluorophore having an excitation peak between 640 nm and 660 nm.

In some embodiments, the detected complex comprises at least 2 detectably labeled probes crosslinked to the second nucleic acid strand. In some embodiments, the detected complex comprises at least 2, 3, 4, 5, 6, or more detectably labeled probes crosslinked to the second nucleic acid strand. In some embodiments, the detected complex comprises between 2 and 20 detectably labeled probes crosslinked to the second nucleic acid strand. In some embodiments, the detected complex comprises at least 2, 3, 4, 5, 6, or more detectably labeled probes crosslinked to the second nucleic acid strand. In some embodiments, the detected complex comprises at least 2, 3, 4, 5, 6, or more detectably labeled probes crosslinked to the second nucleic acid strand. In some embodiments, the detected complex comprises at least 10, at least 20, at least 30, or at least 40 detectably labeled probe bound in the bNA structure. In some embodiments, the detected signal is the signal of at least 10, at least 20, at least 30, or at least 40 detectably labeled probes bound in the bNA structure.

In some embodiments, the kit further comprises any of the first nucleic acid strands, second nucleic acid strands, or detectably labeled probes described herein. In some embodiments, the kit further comprises one or more probes or probe sets for generating one or more target nucleic acid molecules associated with analytes in the sample, such as circularizable probes or probe sets for generating rolling circle amplification products associated with nucleic acid or non-nucleic acid analytes in the biological sample (e.g., circularizable probes or probe sets that hybridize to nucleic acid analytes or hybridization, ligation, or extension products thereof in the biological sample, and/or circularizable probes or probe sets that hybridize to oligonucleotide reporters in labeling agents (such as antibodies) that bind to non-nucleic acid analytes in the biological sample). In some embodiments, the kit further comprises any of the labeling agents described herein (e.g., in Section II). In some embodiments, the kit further comprises a plurality of differently labeled pools of assembled bNA complexes. In some embodiments, each pool of assembled bNA complexes is labeled with a different detectable moiety from one or more other pools of assembled bNA complexes. In some embodiments, each pool of assembled bNA complexes is labeled with a fluorophore of a different fluorescent color. The assembled bNA complexes within the same pool can share the same fluorophore or are labeled with fluorophores of the same fluorescent color, but a pool of assembled bNA complexes can be distinguished from another pool based on a unique fluorophore from among multiple different fluorophores.

In some embodiments, the kit further comprises one or more labeling agents (e.g., an antibody conjugated to oligonucleotide reporter). In some embodiments, the oligonucleotide reporter comprises an amplifier probe hybridization region. In some embodiments, the oligonucleotide reporter comprises an adapter hybridization region. In some embodiments, the oligonucleotide reporter comprises a target sequence for a primary probe.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, modified nucleotides, reagents for additional assays.

### VII. Opto-Fluidic Instruments for Analysis of Biological Samples

Provided herein is an instrument having integrated optics and fluidics modules (an “opto-fluidic instrument” or “opto-fluidic system”) for detecting target molecules (e.g., nucleic acids, proteins, antibodies, etc.) in biological samples (e.g., one or more cells or a tissue sample) as described herein. In an opto-fluidic instrument, the fluidics module is configured to deliver one or more reagents (e.g., detectably labeled probes) to the biological sample and/or remove spent reagents therefrom. Additionally, the optics module is configured to illuminate the biological sample with light having one or more spectral emission curves (over a range of wavelengths) and subsequently capture one or more images of emitted light signals from the biological sample during one or more probing cycles using bNA complexes (e.g., as described in Section II). In various embodiments, the captured images may be processed in real time and/or at a later time to determine the presence of the one or more target molecules in the biological sample, as well as three-dimensional position information associated with each detected target molecule. Additionally, the opto-fluidics instrument includes a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to analyze one or more target molecules (e.g., any of the analytes described in Section III) in their naturally occurring place (i.e., in situ) within the biological sample. For example, an opto-fluidic instrument may be an in-situ analysis system used to analyze a biological sample and detect target molecules (e.g., analytes) including but not limited to DNA, RNA, proteins, antibodies, and/or the like.

It is to be noted that, although the above discussion relates to an opto-fluidic instrument that can be used for in situ target molecule detection via probe hybridization, the discussion herein equally applies to any opto-fluidic instrument that employs any imaging or target molecule detection technique. That is, for example, an opto-fluidic instrument may include a fluidics module that includes fluids needed for establishing the experimental conditions required for the probing of target molecules in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

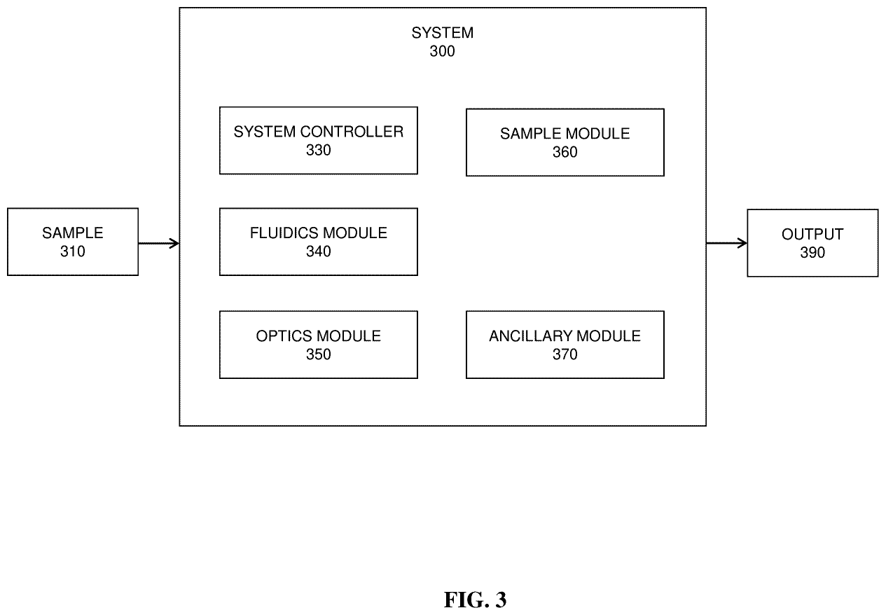

FIG. 3 shows an example workflow of analysis of a biological sample 310 (e.g., cell or tissue sample) using an opto-fluidic instrument or system 300, according to various embodiments. In various embodiments, the sample 310 can be a biological sample (e.g., a tissue) that includes molecules such as DNA, RNA, proteins, antibodies, etc. For example, the sample 310 can be a sectioned tissue that is treated to access the RNA thereof for labeling with probes and bNA complexes described herein (e.g., in Section II). Ligation of the probes may generate a circular probe which can be enzymatically amplified and bound with detectably labeled probes, which can create bright signal that is convenient to image and has a high signal-to-noise ratio.

In various embodiments, the sample 310 may be placed in the opto-fluidic instrument 120 for analysis and detection of the molecules in the sample 310. In various embodiments, the opto-fluidic instrument or system 300 can be a system configured to facilitate the experimental conditions conducive for the detection of the target molecules. For example, the opto-fluidic instrument 320 can include a fluidics module 340, an optics module 350, a sample module 360, and an ancillary module 170, and these modules may be operated by a system controller 330 to create the experimental conditions for the probing of the molecules in the sample 310 by selected probes (e.g., circularizable DNA probes), as well as to facilitate the imaging of the probed sample (e.g., by an imaging system of the optics module 350). In various embodiments, the various modules of the opto-fluidic instrument 320 may be separate components in communication with each other, or at least some of them may be integrated together.

In various embodiments, the sample module 360 may be configured to receive the sample 310 into the opto-fluidic instrument or system 300. For instance, the sample module 360 may include a sample interface module (SIM) that is configured to receive a sample device (e.g., cassette) onto which the sample 310 can be deposited. That is, the sample 310 may be placed in the opto-fluidic instrument or system 300 by depositing the sample 310 (e.g., the sectioned tissue) on a sample device that is then inserted into the SIM of the sample module 360. In some instances, the sample module 360 may also include an X-Y stage onto which the SIM is mounted. The X-Y stage may be configured to move the SIM mounted thereon (e.g., and as such the sample device containing the sample 310 inserted therein) in perpendicular directions along the two-dimensional (2D) plane of the opto-fluidic instrument or system 300.

The experimental conditions that are conducive for the detection of the molecules in the sample 310 may depend on the target molecule detection technique that is employed by the opto-fluidic instrument or system 300. For example, in various embodiments, the opto-fluidic instrument or system 300 can be a system that is configured to detect molecules in the sample 310 via hybridization of probes. In such cases, the experimental conditions can include molecule hybridization conditions that result in the intensity of hybridization of the target molecule (e.g., nucleic acid) to a probe (e.g., oligonucleotide) being significantly higher when the probe sequence is complementary to the target molecule than when there is a single-base mismatch. The hybridization conditions include the preparation of the sample 310 using reagents such as washing/stripping reagents, hybridizing reagents, etc., and such reagents may be provided by the fluidics module 140.

In various embodiments, the fluidics module 340 may include one or more components that may be used for storing the reagents, as well as for transporting said reagents to and from the sample device containing the sample 310. For example, the fluidics module 340 may include reservoirs configured to store the reagents, as well as a waste container configured for collecting the reagents (e.g., and other waste) after use by the opto-fluidic instrument or system 300 to analyze and detect the molecules of the sample 310. Further, the fluidics module 340 may also include pumps, tubes, pipettes, etc., that are configured to facilitate the transport of the reagent to the sample device (e.g., and as such the sample 310). For instance, the fluidics module 340 may include pumps (“reagent pumps”) that are configured to pump washing/stripping reagents to the sample device for use in washing/stripping the sample 310 (e.g., as well as other washing functions such as washing an objective lens of the imaging system of the optics module 350).

In various embodiments, the ancillary module 370 can be a cooling system of the opto-fluidic instrument 320, and the cooling system may include a network of coolant-carrying tubes that are configured to transport coolants to various modules of the opto-fluidic instrument or system 300 for regulating the temperatures thereof. In such cases, the fluidics module 340 may include coolant reservoirs for storing the coolants and pumps (e.g., “coolant pumps”) for generating a pressure differential, thereby forcing the coolants to flow from the reservoirs to the various modules of the opto-fluidic instrument 320 via the coolant-carrying tubes. In some instances, the fluidics module 340 may include returning coolant reservoirs that may be configured to receive and store returning coolants, e.g., heated coolants flowing back into the returning coolant reservoirs after absorbing heat discharged by the various modules of the opto-fluidic instrument or system 300. In such cases, the fluidics module 340 may also include cooling fans that are configured to force air (e.g., cool and/or ambient air) into the returning coolant reservoirs to cool the heated coolants stored therein. In some instance, the fluidics module 340 may also include cooling fans that are configured to force air directly into a component of the opto-fluidic instrument or system 300 so as to cool said component. For example, the fluidics module 340 may include cooling fans that are configured to direct cool or ambient air into the system controller 330 to cool the same.

As discussed above, the opto-fluidic instrument 320 may include an optics module 350 which include the various optical components of the opto-fluidic instrument or system 300, such as but not limited to a camera, an illumination module (e.g., LEDs), an objective lens, and/or the like. The optics module 350 may include a fluorescence imaging system that is configured to image the fluorescence emitted by the probes (e.g., oligonucleotides) in the sample 310 after the probes are excited by light from the illumination module of the optics module 350.

In some instances, the optics module 350 may also include an optical frame onto which the camera, the illumination module, and/or the X-Y stage of the sample module 360 may be mounted.

In various embodiments, the system controller 330 may be configured to control the operations of the opto-fluidic instrument or system 300 (e.g., and the operations of one or more modules thereof). In some instances, the system controller 330 may take various forms, including a processor, a single computer (or computer system), or multiple computers in communication with each other. In various embodiments, the system controller 330 may be communicatively coupled with data storage, set of input devices, display system, or a combination thereof. In some cases, some or all of these components may be considered to be part of or otherwise integrated with the system controller 330, may be separate components in communication with each other, or may be integrated together. In other examples, the system controller 330 can be, or may be in communication with, a cloud computing platform.

In various embodiments, the opto-fluidic instrument or system 300 may analyze the sample 310 and may generate the output 390 that includes indications of the presence of the target molecules in the sample 310. For instance, with respect to the example embodiment discussed above where the opto-fluidic instrument or system 300 employs a hybridization technique for detecting molecules, the opto-fluidic instrument or system 300 may cause the sample 310 to undergo successive rounds of detectably labeled probe hybridization (e.g., using two or more sets of fluorescent probes, where each set of fluorescent probes is excited by a different color channel) and be imaged to detect target molecules in the probed sample 310. In such cases, the output 390 may include optical signatures (e.g., a codeword) specific to each gene, which allow the identification of the target molecules.

### VIII. Applications

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### IX. Terminology

Unless defined otherwise, all terms of art, notations and other technical and

scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer as used herein, in some embodiments, is an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

In some instances, “ligation” refers to the formation of a covalent bond or linkage between the termini of two or more nucleic acids, e.g., oligonucleotides and/or polynucleotides, in a template-driven reaction. The nature of the bond or linkage may vary widely and the ligation, in some embodiments, is carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: Assembly of Branched DNA Structures Via Click Chemistry

This example describes a method for the design and construction of a crosslinked branched nucleic acid (bNA) structure through click chemistry, wherein the bNA structure comprises a first nucleic acid strand and a plurality of second nucleic acid strands. Exemplary nucleic acid strand designs are shown in FIG. 1A. Assembly of the bNA structure is achieved through copper-free click-chemistry catalyzed reactions conjugating the azide- and DBCO-modified bases via a triazole moiety.

The first nucleic acid strand is designed to comprise an adapter hybridization region that hybridizes to an adapter region in a target nucleic acid or in a probe or probe set hybridized to target nucleic acid in the biological sample, and a plurality of branch points comprising azide-modified bases. The branch points comprising azide-modified bases are incorporated into the first nucleic acid strand such that a distance of between 10 and 20 nucleotide residues separates each branch point. The probe or probe set that the adapter hybridization region in the first nucleic acid hybridizes to can comprise, for example, a primary probe that binds to the target nucleic acid such as RNA.

The second nucleic acid strands are designed to comprise a single DBCO-modified base and a plurality of branch points comprising azide modified bases. The branch points comprising azide-modified bases are incorporated into the second nucleic acid strands such that a distance of between 10 and 20 nucleotide residues separates each branch point.

Detectably labeled probes are designed to comprise a single DBCO-modified base and a detectable moiety. The detectable moiety can comprise, for example, a fluorophore such as Alexa Fluor 488.

An exemplary assembly protocol for the construction of the bNA structure is shown in FIG. 1B. To begin the assembly of the bNA structure, the second nucleic acid strands are mixed with the detectably labeled probes in a ratio consistent with the number of branch points present in the second nucleic acid strand. For example, a second nucleic acid strand containing four branch points will require a ratio of 1:4 second nucleic acid strands to detectably labeled probes. Conjugation of the azide modified bases in the branch points within the second nucleic acid with the DBCO-modified base present in the detectably labeled probe is catalyzed through copper-free click-chemistry.

Following the first copper-free click-chemistry reaction, the covalently linked second nucleic acid strand and detectably labeled probes are mixed with the first nucleic acid strands. In this reaction, the azide-modified bases in the branch points within the first nucleic acid strand are covalently linked to the DBCO-modified base in the second nucleic acid strand through a triazole moiety. This second reaction results in a fully assembled bNA structure comprising a single first nucleic acid strand containing an adapter hybridization region that hybridizes to an adapter region in a target nucleic acid or in a probe or probe set conjugated to a plurality of detectably labeled probes. In this example design, a first nucleic acid strand containing three branch points and a second nucleic acid containing four branch points result in an assembled bNA structure containing 12 detectably labeled probes, thereby significantly improving the detectable signal generated per probes.

For a method of multiplex detection, crosslinked bNA structures comprising different detectable moieties (e.g., different color fluorophores) can be prepared separately as described above, and then pooled to provide a bNA structure mix. In an example, the bNA structure mix can comprise 4 different species of bNAs comprising different color fluorophores: e.g., a red, yellow, green, and blue bNA. An exemplary method to analyze a biological sample using the crosslinked bNA structures is provided below.

A tissue section is prepared and adhered to a transparent substrate. The tissue section is contacted with a pool of primary probes are designed to hybridize to target nucleic acids within the biological sample. The probe hybridization mix is then removed, and the chambers are washed to remove unhybridized probes. The probes comprise barcode sequences corresponding to their respective target nucleic acids.

The first bNA structure mix (comprising the 4 different species of bNA) is added to the tissue section in hybridization buffer, and incubated to allow hybridization (e.g., for 1 h at 20-37° C.). The sections are then washed to remove unhybridized bNA. An example of a bNA structure hybridized to a primary probe and target nucleic acid is shown in FIG. 1C.

Following the detection of the first bNA structures, the first bNA structures can be removed (e.g., by stripping), or the detectable moieties can be removed by cleavage or quenched to prevent subsequent detection of the detectable moieties. Successive rounds of hybridizing bNA structures can be performed, depending on the number of analytes to be assessed. This method of successive hybridization can impart a series of signal codes (e.g., a series of fluorescent colors) corresponding to a target nucleic acid, allowing for multiplexed analysis of the biological sample.

### Example 2: Assembly of Branched DNA Structures Via Crosslinking

This example describes a method for the design, construction, and use of a crosslinked bNA structure through photocrosslinkable nucleotides, wherein the bNA structure comprises a first nucleic acid strand and a plurality of second nucleic acid strands. Exemplary nucleic acid strand designs are shown in FIG. 2A. Assembly of the bNA structure is achieved by hybridization between the nucleic acid strands. Crosslinking of the hybridized bNA structure is achieved by photocrosslinking. For example, a cyanovinylcarbazole nucleoside analogue (^{CNV}K) can be incorporated into a nucleic acid strand and a short pulse of irradiation at 366 nm results in efficient interstrand cross-linking with adjacent pyrimidine bases. Importantly, this interstrand crosslink can be reversed via a 3 minute irradiation at 312 nm, providing significant temporal control over bNA structure stability.

The first nucleic acid strand is designed to comprise an adapter hybridization region that hybridizes to an adapter region on a target nucleic acid or in a probe or probe set hybridized to target nucleic acid in the biological sample, and a plurality of branch hybridization regions.

The second nucleic acid strand is designed to comprise a branch hybridization region that hybridizes to the branch regions in the first nucleic acid strand, and a plurality of reporter regions. The branch hybridization region and reporter regions are designed to contain a crosslinkable moiety. For example, a ^{CNV}K modification is incorporated into the branch hybridization region and reporter regions at residues opposite of pyrimidine nucleotides in the first nucleic acid and detectably labeled probes, respectively.

The detectably labeled probe is designed to comprise a reporter hybridization region and a detectable moiety. The detectable moiety can comprise, for example, a fluorophore such as Alexa Fluor 488.

A schematic illustrating the assembly and crosslinking of the bNA structure is shown in FIG. 2B. To begin the assembly of the bNA structure, first nucleic acid strands, second nucleic acid strands, and detectably labeled probes are mixed in a single hybridization reaction. The relative quantities of nucleic acids strands depends on the number of branch hybridization and reporter hybridization regions included in the first nucleic acid strands and second nucleic acid strands, respectively. For example, a bNA structure comprising a first nucleic acid strand containing three first nucleic acid strands and second nucleic acid strands containing three reporter regions will be assembled at a ratio of 1 first nucleic acid strand to 3 second nucleic acid strands to 9 detectably labeled probes. Following assembly of the bNA structure via hybridization between the first nucleic acid strand, second nucleic acid strands, and detectably labeled probes, the bNA structure is crosslinked by irradiation. For example, a bNA structure containing ^{CNV}K modified nucleotides in the second nucleic acid strand will be crosslinked via a 30 second irradiation at 366 nm. The bNA structure will then contain crosslinks between the first nucleic acid strand, second nucleic acid strands, and detectably labeled probes, providing increased thermostability.

After formation of the crosslinked bNA structure, the crosskinked bNA structure can be used to detect target nucleic acids in a biological sample as described in Example 1 above. If desired, crosslinked bNA structures corresponding to different detectable moieties can be pooled and used in a method of sequential hybridization for multiplex detection of target nucleic acids as described in Example 1 above. Optionally, the crosslinking to form the bNA structures can be reversed by irradiating the tissue section at a wavelength that reverses the crosslinks before or during the wash step (e.g., during a DAPI imaging step).

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1-80. (canceled)

81. A method for analyzing a biological sample, comprising:
(a) contacting the biological sample with a crosslinked branched nucleic acid (bNA) structure comprising a first nucleic acid strand and a plurality of second nucleic acid strands,
wherein the first nucleic acid strand comprises: (i) an adapter hybridization region that hybridizes to an adapter region in a target nucleic acid or in a probe or probe set hybridized to a target nucleic acid in the biological sample, and (ii) a plurality of branch hybridization regions (BHR),
wherein each of the second nucleic acid strands comprises a complementary branch hybridization region (BHR′) and a detectable label or an overhang region comprising one or more reporter regions,
wherein BHR′ hybridizes to BHR, wherein at least a subset of the second nucleic acid strands are covalently attached to the first nucleic acid strand via an interstrand crosslink in the branch hybridization region; and
(b) detecting the hybridized bNA structure at a location in the biological sample by detecting the detectable label or the one or more reporter regions, thereby detecting the target nucleic acid at the location in the biological sample.

82. The method of claim 81, wherein the adapter region is in a probe or probe set, and wherein the method comprises contacting the biological sample with the probe or probe set,
wherein the probe or probe set comprises (i) a recognition sequence that hybridizes to the target nucleic acid in the biological sample, and (ii) an overhang region comprising the adapter region.

83. The method of claim 81, wherein each of the second nucleic acid strands comprises an overhang region comprising a plurality of reporter regions,
wherein the bNA structure comprises a plurality of detectably labeled probes hybridized to the reporter regions via complementary reporter hybridization regions, optionally wherein at least a subset of the reporter regions are covalently attached to the hybridized detectably labeled probes via an interstrand crosslink.

84. The method of claim 81, wherein prior to the contacting in (a), the method comprises generating the crosslinked bNA structure by providing a mixture comprising the first nucleic acid strand and the plurality of second nucleic acid strands and allowing the branch hybridization regions (BHR) to hybridize to the complementary branch hybridization region (BHR′), and irradiating the mixture to generate interstrand crosslinks between the branch hybridization regions (BHR) and the complementary branch hybridization region (BHR′).

85. The method of claim 84, wherein generating the crosslinked bNA structure further comprises contacting the second nucleic acid strand with the plurality of detectably labeled probes and allowing the detectably labeled probes to hybridize to the reporter regions, and irradiating the mixture to generate interstrand crosslinks between the reporter regions and the hybridized detectably labeled probes.

86. The method of claim 81, wherein the interstrand crosslink between the branch hybridization region (BHR) and the complementary branch hybridization region (BHR′) was generated by irradiating a crosslinkable moiety comprised by the branch hybridization region (BHR) and/or the complementary branch hybridization region (BHR′).

87. The method of claim 81, wherein the interstrand crosslinks occur at internal nucleotides of the BHR and BHR′.

88. The method of claim 81, wherein the interstrand crosslink between the reporter region and the complementary reporter hybridization region was generated by irradiating a crosslinkable moiety comprised by the reporter region and/or the complementary reporter hybridization region.

89. The method of claim 81, wherein the interstrand linkage is generated by UV-irradiation of a crosslinkable moiety, wherein the crosslinkable moiety is a modified nucleoside in the first nucleic acid strand, second nucleic acid strands, and/or detectably labeled probes.

90. The method of claim 89, wherein the method comprises irradiating the mixture to photo-activate the crosslinkable moiety.

91. The method of claim 85, wherein the mixture is irradiated using a 350-400 nm wavelength of light.

92. The method of claim 84, wherein the crosslinkable moiety is a 3-cyanovinylcarbazole (^{CNV}K) nucleoside.

93. The method of claim 81, wherein the interstrand crosslink is reversible, and wherein after detecting the hybridized bNA structure, the method comprises irradiating the biological sample to reverse one or more of the interstrand linkages.

94. The method of claim 93, wherein irradiating the biological sample to reverse one or more of the interstrand linkages comprises irradiating the sample with a wavelength between about 300 nm and about 320 nm.

95. The method of claim 94, wherein detecting the bNA structure comprises imaging the biological sample at one or more excitation wavelengths between about 488 nm and about 647 nm.

96. The method of claim 81, wherein the method comprises washing the biological sample to remove the bNA structure after the detecting in (b).

97. The method of claim 81, wherein the bNA structure is a first bNA structure, and detecting the first bNA structure comprises detecting a first signal of a signal code assigned to the target nucleic acid,
wherein after detecting the hybridized first bNA structure at a location in the biological sample, the method comprises:
(c) removing and/or cleaving the first bNA structure,
(d) contacting the biological sample with a second bNA structure, wherein the second bNA structure binds directly or indirectly to the target nucleic acid, and
(e) detecting the second bNA structure, wherein detecting the second bNA structure comprises detecting a second signal of the signal code assigned to the target nucleic acid.

98. The method of claim 97, wherein removing the first bNA structure comprises de-crosslinking the first bNA structure.

99. The method of claim 81, wherein the target nucleic acid is an oligonucleotide reporter in a labeling agent that binds to a protein analyte.

100. The method of claim 81, wherein the target nucleic acid is RNA or a product thereof.
